(19) United States
(12) Patent Application Publication
(10) Pub. No.: US 2026/0167755 A1
(43) Pub. Date: Jun. 18, 2026

(54) Radiation-Sensitive Composition, Resist Pattern Formation Method, Acid-Generator, and Compound

(71) Applicant: JSR CORPORATION, Tokyo (JP)

(72) Inventors: Natsuko KINOSHITA, Tokyo (JP); Takuhiro TANIGUCHI, Tokyo (JP)

(73) Assignee: JSR CORPORATION, Tokyo (JP)

(21) Appl. No.: 18/720,792

(22) PCT Filed: Nov. 7, 2022
(86) PCT No.: PCT/JP2022/041361

§ 371 Date: Jun. 17, 2024

(30) Foreign Application Priority Data
Dec. 21, 2021 (JP) 2021-207252

(51) Int. Cl. C08F 220/24

(52) U.S. Cl. CPC C08F 220/24; C07C 2601/14

(57) ABSTRACT

A radiation-sensitive composition contains: a polymer having an acid-releasable group and at least one compound (b) selected from the group consisting of a compound represented by formula (1) and a compound represented by formula (2). In formula (1), R¹ represents a C1 to C20 monovalent organic group. R² represents a single bond or a C1 to C20 divalent group which bonds to N⁻ in formula (1) via —CR⁴R⁵— or an aromatic ring. M^{a+} represents an a-valent cation. In formula (2), R⁷ represents a group having a partial structure in which an iodine atom is bonded to an aromatic ring. M^{b+} represents a b-valent cation.

### CROSS-REFERENCE TO RELATED APPLICATIONS

The present application claims the benefit of Japanese Patent Application No. 2021-207252 filed Dec. 21, 2021, the disclosure of which is incorporated herein by reference.

The present disclosure relates to a radiation-sensitive composition, to a method for forming a resist pattern (hereinafter may also be referred to as a “resist pattern formation method”), to an acid-generator, and to a compound.

### TECHNICAL FIELD

### Background Art

In a lithography technique employed in production of various electronic devices including semiconductor devices and liquid crystal devices, a process target formed of a radiation-sensitive composition is irradiated with a far-UV ray (e.g., ArF excimer laser light), an extreme UV (EUV) ray, an electron beam, or the like, to thereby generate acid in a light-exposed part. Through chemical reaction involving the generated acid, difference in dissolution rate with respect to a developer is provided between the light-exposed part and the light-unexposed part. Thus, a resist pattern is formed on a substrate.

Meanwhile, structures of such electronic devices have been further miniaturized steeply. Under such circumstances, further fine resist patterns are required in lithography steps. In addition, in order to satisfy the requirement of further fine resist patterns, various studies have been done for improving resolution of a radiation-sensitive composition employed in lithographic micro-processing, rectangularity of a resultant resist pattern, and the like.

For improving the pattern shape of the resist pattern, a key factor is to gain a sufficiently high contrast in terms of solubility in a developer between a light-exposed part and a light-unexposed part. Thus, hitherto, a quencher (an acid diffusion suppressor) has been employed for suppressing diffusion of an acid to a light-unexposed part and controlling the solubility of the unexposed part in a developer (see, for example, Patent Documents 1 to 3). Patent Documents 1 to 3 disclose, as such a quencher, a sulfonium salt which can generate iodobenzoic acid and an onium salt having an N-deficient carbonylsulfonamide structure.

### PRIOR ART DOCUMENTS

### Patent Documents

Patent Document 1: Japanese Patent Application Laid-Open (kokai) No. 2017-219836
Patent Document 2: Japanese Patent Application Laid-Open (kokai) No. 2019-211751
Patent Document 3: Japanese Patent Application Laid-Open (kokai) No. 2020-098330

### SUMMARY OF THE INVENTION

### Problems to be Solved by the Invention

When a radiation-sensitive composition has considerably high solubility in a developer, the pattern portion may possibly be dissolved in the developer during development, resulting in undesired development failures such as breaking.

Meanwhile, in a rapid progress in further process shrinkage of resist patterns in recent years, attempts have been made to form a pattern having, for example, a line width of 40 nm or less. Thus, the radiation-sensitive composition also employed in formation of such fine resist patterns must provide a suitable resist pattern having excellent lithographic characteristics such as LWR (line width roughness) performance by a small dose (i.e., at high sensitivity).

The disclosure has been made in view of the aforementioned problems. Thus, an object of the present disclosure is to provide a high-sensitive radiation-sensitive composition which can form a resist pattern exhibiting excellent LWR performance, wherein generation of development failure is suppressed. Another object is to provide a method for forming such a resist pattern.

### Means for Solving the Problems

According to the present disclosure, the following means are provided.

[1]A radiation-sensitive composition includes: a polymer having an acid-releasable group, and at least one compound (b) selected from the group consisting of a compound represented by formula (1) and a compound represented by formula (2).

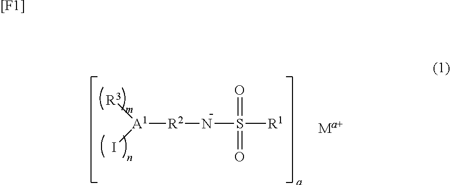

(In formula (1), R¹ represents a C1 to C20 monovalent organic group; R² represents a single bond or a C1 to C20 divalent group which bonds to N⁻ in formula (1) via —CR⁴R⁵— or an aromatic ring; each of R⁴ and R⁵ independently represents a hydrogen atom, a C1 to C3 monovalent hydrocarbon group, or —COOR⁶; R⁶ represents a C1 to C6 monovalent hydrocarbon group; A¹ represents a group formed by removing (m+n+1) hydrogen atoms from an aromatic ring; R³ represents a monovalent substituent other than an iodine atom; m is an integer of 0 or greater; n is an integer of 1 or greater; when m is ≥2, a plurality of groups “R³” are identical to or different from one another; M^{a+} represents an a-valent cation; and a is 1 or 2.)

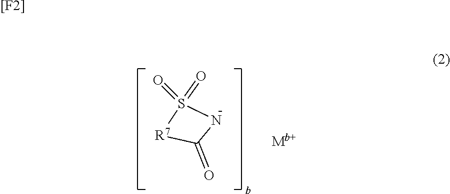

(In formula (2), R⁷ represents a group represented by formula (r-1), a group represented by formula (r-2), or a group represented by formula (r-3); M^{b+} represents a b-valent cation; and b is 1 or 2.)

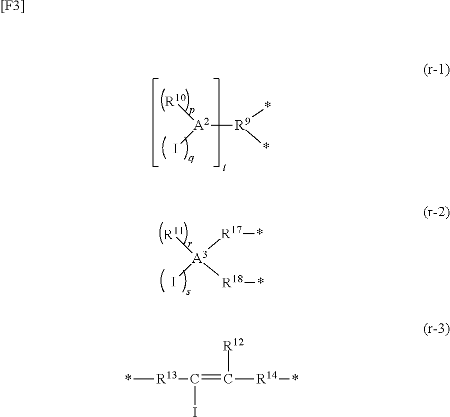

(In formula (r-1), R⁹ represents a C1 to C10 (t+2)-valent hydrocarbon group, a C1 to C10 (t+2)-valent organic group formed by substituting any methylene group of the hydrocarbon group with —O—, —S—, —NR¹⁵—, or a carbonyl group, or a (t+2)-valent group formed by substituting any hydrogen atom of the hydrocarbon group or the (t+2)-valent organic group with a substituent; R¹⁵ represents a hydrogen atom or a C₁ to C₅ monovalent hydrocarbon group; A² represents a group formed by removing (p+q+1) hydrogen atoms from an aromatic ring; R¹⁰ represents a monovalent substituent other than an iodine atom; p is an integer of 0 or greater; q is an integer of 1 or greater; when p is 2, a plurality of groups “R¹⁰” are identical to or different from one another; t is 1 or 2; when t is 2, a plurality of groups “A²” are identical to or different from one another, and a plurality of groups “R¹⁰” are identical to or different from one another; and “*” represents a chemical bond;
in formula (r-2), A³ represents a group formed by removing (r+s+2) hydrogen atoms from an aromatic ring; R¹¹ represents a monovalent substituent other than an iodine atom; each of R¹⁷ and R¹⁸ independently represents a single bond, —O—, —S—, —NR¹⁹—, a carbonyl group, a C1 to C3 divalent chain hydrocarbon group, or a C1 to C6 divalent group formed by substituting any methylene group of the chain hydrocarbon group with —O—, —S—, —NR¹⁹—, or a carbonyl group; R¹⁹ represents a hydrogen atom or a C1 to C5 monovalent hydrocarbon group; r is an integer of 0 or greater; s is an integer of 1 or greater; when r is 2, a plurality of groups “R¹¹” are identical to or different from one another; and “*” represents a chemical bond; and
in formula (r-3), R¹² represents a hydrogen atom, an iodine atom, or a C1 to C5 monovalent hydrocarbon group; each of R¹³ and R¹⁴ independently represents a single bond, —O—, —S—, —NR¹⁶—, a carbonyl group, a C1 to C3 divalent chain hydrocarbon group, or a C1 to C6 divalent group formed by substituting any methylene group of the chain hydrocarbon group with —O—, —S—, —NR¹⁶—, or a carbonyl group; R¹⁶ represents a hydrogen atom or a C1 to C5 monovalent hydrocarbon group; and “*” represents a chemical bond.)
   [2]A resist pattern formation method includes: a step of forming a resist film on a substrate by use of a radiation-sensitive composition as recited in [1] above, a step of exposing the resist film to light, and a step of developing the light-exposed resist film.
   [3] An acid-generator represented by the aforementioned formula (1).
   [4] An acid-generator represented by the aforementioned formula (2).
   [5]A compound represented by the aforementioned formula (1)
   [6]A compound represented by the aforementioned formula (2)

### Advantageous Effects of the Invention

Since the radiation-sensitive composition of the present disclosure has high sensitivity, a more favorable resist pattern can be formed by a small light exposure dose. In addition, the radiation-sensitive composition of the present disclosure can form a resist pattern exhibiting excellent LWR performance, wherein generation of development failure is suppressed.

### MODES FOR CARRYING OUT THE INVENTION

### <Radiation-Sensitive Composition>

The radiation-sensitive composition of the present disclosure (hereinafter may also be referred to simply as “the present composition”) contains a polymer [A] and an acid-generator [B]. The present composition may further contain, as a suitable component, one or more members of a solvent [E], and a high-fluorine content polymer [F]. These components will next be described in detail.

As used herein, the term “hydrocarbon group” encompasses a chain hydrocarbon group, an alicyclic hydrocarbon group, and an aromatic hydrocarbon group. The term “chain hydrocarbon group” refers to a linear-chain hydrocarbon group or a branched hydrocarbon group including one which is composed of only a chain structure and no ring structure. However, the chain hydrocarbon group may be saturated or unsaturated. The term “alicyclic hydrocarbon group” refers to a hydrocarbon group which contains only an alicyclic hydrocarbon moiety as a ring structure and contains no aromatic ring structure. However, the alicyclic hydrocarbon group is not necessarily formed only of an alicyclic hydrocarbon moiety and may contain a chain structure as a partial structure. The term “aromatic hydrocarbon group” refers to a hydrocarbon group which contains an aromatic ring structure as a ring structure. However, the aromatic hydrocarbon group is not necessarily formed only of an aromatic ring structure and may contain a chain structure or an alicyclic hydrocarbon moiety as a partial structure. The term “aromatic ring group” refers to an n-valent group formed by removing n (n is an integer of 1 or greater) hydrogen atoms from a ring portion of a substituted or unsubstituted aromatic ring. The term “organic group” refers to an atomic group formed by removing any hydrogen atom from a carbon-containing compound (i.e., an organic compound).

### <Polymer [A]>

Polymer [A] is a polymer having an acid-releasable group. The polymer [A] includes a structural unit having an acid-releasable group (hereinafter may also be referred to as a “structural unit (I)”) and may further include a structural unit differing from the structural unit (I) (hereinafter may also be referred to as an “additional structural unit”).

### [Structural Unit (I)]

The acid-releasable group refers to a group which can substitute a hydrogen atom of an acidic group such as a carboxy group or a hydroxy group and which is eliminated by the action of acid. When the polymer [A] has an acid-releasable group, the acid-releasable group is released from the present composition via exposure to light to thereby form an acidic group, which modifies the solubility of the polymer component(s) in a developer. As a result, excellent lithographic characteristics can be imparted to the present composition.

No particular limitation is imposed on the structural unit (I), so long as the unit has an acid-releasable group. Examples of the structural unit (I) include structural units represented by the below-described formula (i-1) (hereinafter may also be referred to as “structural units (I-1)”) and structural units represented by the below-described formula (i-2) (hereinafter may also be referred to as “structural units (I-2)”).

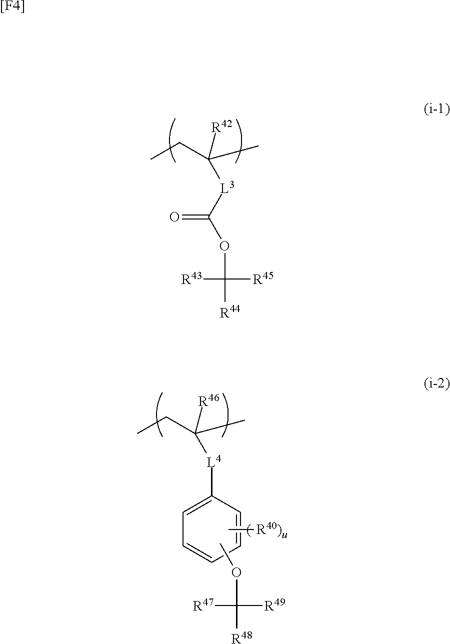

(In formula (i-1), R⁴² represents a hydrogen atom, a fluorine atom, a methyl group, or a trifluoromethyl group; L³ represents a single bond, a substituted or unsubstituted phenylene group, **—COO—Ar¹—, or **—CONH—Ar¹—; Ar¹ represents a substituted or unsubstituted phenylene group; “**” represents a chemical bond to be linked to a carbon atom to which R⁴² is bonded; R⁴³ is a hydrogen atom or a C1 to C20 monovalent hydrocarbon group; each of R⁴⁴ and R⁴⁵ independently represents a C1 to C20 monovalent hydrocarbon group, or R⁴⁴ and R⁴⁵ are linked to form a C3 to C20 alicyclic structure including the carbon atom to which R⁴⁴ and R⁴⁵ are bound; when R⁴³ is a hydrogen atom, at least one of R⁴⁴ and R⁴⁵ represents a monovalent unsaturated hydrocarbon group, or a C3 to C20 unsaturated alicyclic structure including the carbon atom to which R⁴⁴ and R⁴⁵ are bound, the structure being formed by linking R⁴⁴ and R⁴⁵; and hydrogen atoms of each of R⁴³, R⁴⁴, and R⁴⁵ may be partially or totally substituted with a halogen atom; and
in formula (i-2), R⁴⁶ represents a hydrogen atom, a fluorine atom, a methyl group, or a trifluoromethyl group; L⁴ represents a single bond, —COO—, or —CONH—; each of R⁴⁷, R⁴⁸, and R⁴⁹ independently represents a hydrogen atom, a C1 to C20 monovalent hydrocarbon group, or a C1 to C20 monovalent oxyhydrocarbon group; R⁴⁰ represents a hydroxy group, a C1 to C10 monovalent hydrocarbon group, or a C1 to C10 oxyhydrocarbon group; u is an integer of 0 to 4; and hydrogen atoms of each of R⁴⁷, R⁴⁸, and R⁴⁹ may be partially or totally substituted with a halogen atom.)

In the aforementioned formula (i-1), R⁴² is preferably a hydrogen atom or a methyl group, more preferably a methyl group, from the viewpoint of co-polymerizability of a monomer providing the structural unit (I-1). In the aforementioned formula (i-2), from the viewpoint of co-polymerizability of a monomer providing the structural unit (I-2), R⁴⁶ is preferably a hydrogen atom.

Examples of the C1 to C20 monovalent hydrocarbon group represented by each of R⁴³ to R⁴⁵ and R⁴⁷ to R⁴⁹ include a C1 to C20 monovalent chain hydrocarbon group, a C3 to C20 monovalent alicyclic hydrocarbon group, and a C6 to C20 monovalent aromatic hydrocarbon group. Specific examples of the C1 to C20 monovalent chain hydrocarbon group include alkyl groups such as methyl, ethyl, n-propyl, i-propyl, n-butyl, i-butyl, sec-butyl, t-butyl, and pentyl; alkenyl groups such as ethenyl, propenyl, butenyl, and pentenyl; and alkynyl groups such as ethynyl, propynyl, butynyl, and pentynyl.

Examples of the C3 to C20 monovalent alicyclic hydrocarbon group include monocyclic alicyclic saturated hydrocarbon groups such as cyclopropyl, cyclobutyl, cyclopentyl, and cyclohexyl; polycyclic alicyclic saturated hydrocarbon groups such as norbornyl, adamantyl, tricyclodecyl, and tetracyclododecyl; monocyclic alicyclic unsaturated hydrocarbon groups such as cyclopropenyl, cyclobutenyl, cyclopentenyl, and cyclohexenyl; and polycyclic alicyclic saturated hydrocarbon groups such as norbornenyl and tricyclodecenyl.

Examples of the C6 to C20 monovalent aromatic hydrocarbon group include aryl groups such as phenyl, tolyl, xylyl, naphthyl, and anthryl; and aralkyl groups such as benzyl, phenethyl, naphtylmethyl, and anthrylmethyl.

The C3 to C20 alicyclic structure including the carbon atom to which R⁴⁴ and R⁴⁵ are bound, the structure being formed by linking R⁴⁴ and R⁴⁵, may be a saturated structure or an unsaturated structure. Examples of the alicyclic structure include monocyclic alicyclic structures such as a cyclopropane structure, a cyclobutane structure, a cyclopentane structure, a cyclopentene structure, a cyclohexane structure, a cyclohexene structure, a cycloheptane structure, and a cyclooctane structure; and polycyclic alicyclic structures such as a norbornane structure, an adamantane structure, a tricyclodecane structure, and a tetracyclododecane structure.

When at least one of R⁴⁴ and R⁴⁵ is a monovalent unsaturated hydrocarbon group, examples of the monovalent unsaturated hydrocarbon group include a C1 to C20 monovalent unsaturated chain hydrocarbon group, a C3 to C20 monovalent alicyclic unsaturated hydrocarbon group, and a C6 to C20 monovalent aromatic hydrocarbon group. Specific examples thereof include the same groups as exemplified in relation to the aforementioned hydrocarbon groups.

Examples of the C1 to C20 monovalent oxyhydrocarbon group represented by any of R⁴⁷ to R⁴⁹ include groups formed by incorporating an oxygen atom into the chemical bond side end of any of C1 to C20 monovalent hydrocarbon groups represented by the aforementioned R⁴³ to R⁴⁵ and R⁴⁷ to R⁴⁹, as exemplified above. R⁴⁷ to R⁴⁹ are preferably a chain hydrocarbon group and a cycloalkyloxy group.

In the substituted phenylene group represented by L³ or Ar¹, examples of the substituent incorporated into the phenylene group include a hydroxy group, a C1 to C10 monovalent hydrocarbon group, and a C1 to C10 oxyhydrocarbon group.

Specific examples of the structural unit (I-1) include structural units represented by the following formulas.

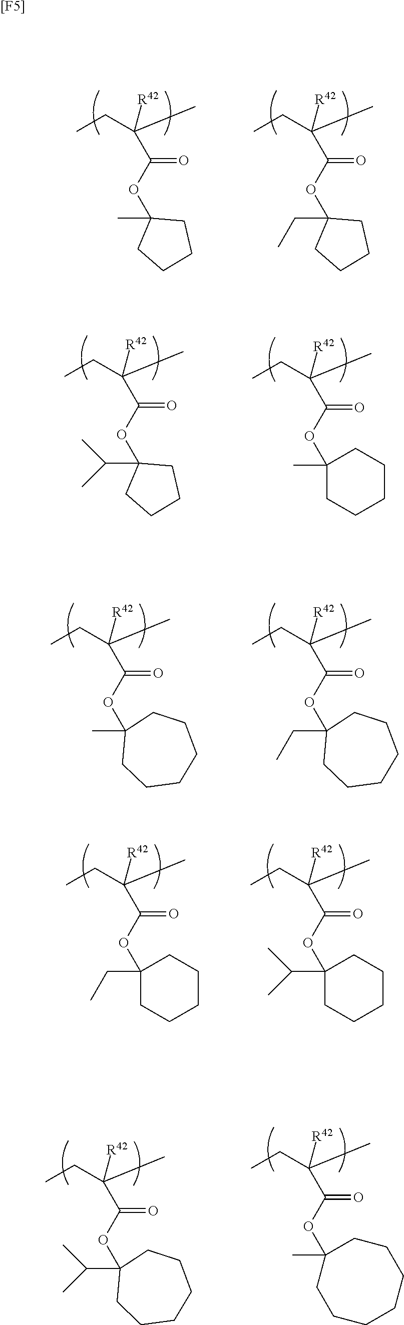

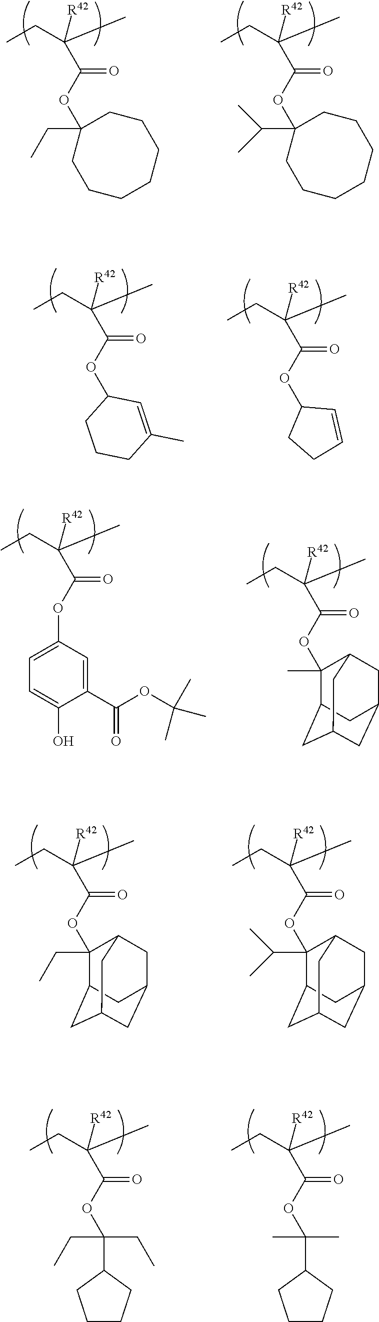

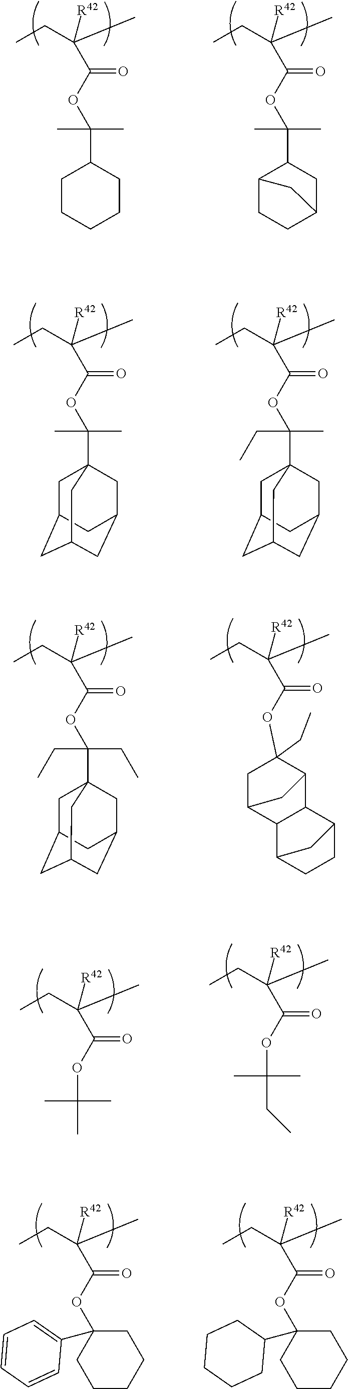

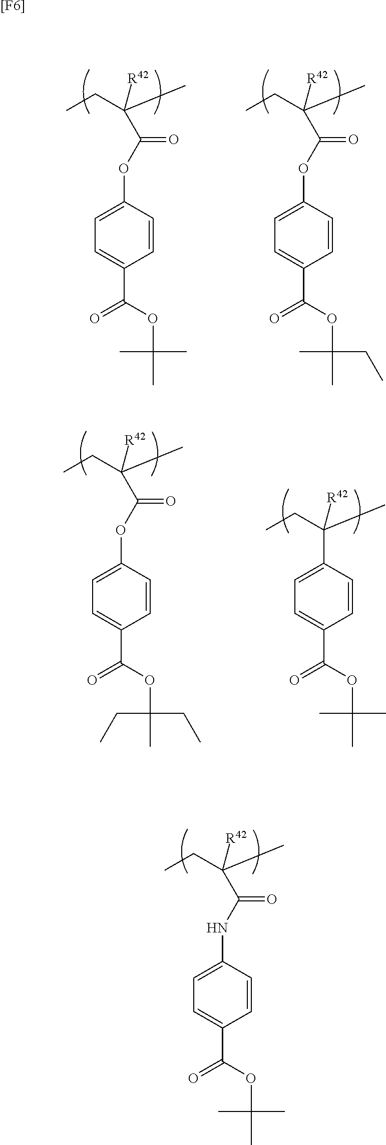

(In the above formulas, R⁴² represents a hydrogen atom, a fluorine atom, a methyl group, or a trifluoromethyl group.)

Specific examples of the structural unit (I-2) include structural units represented by the following formulas.

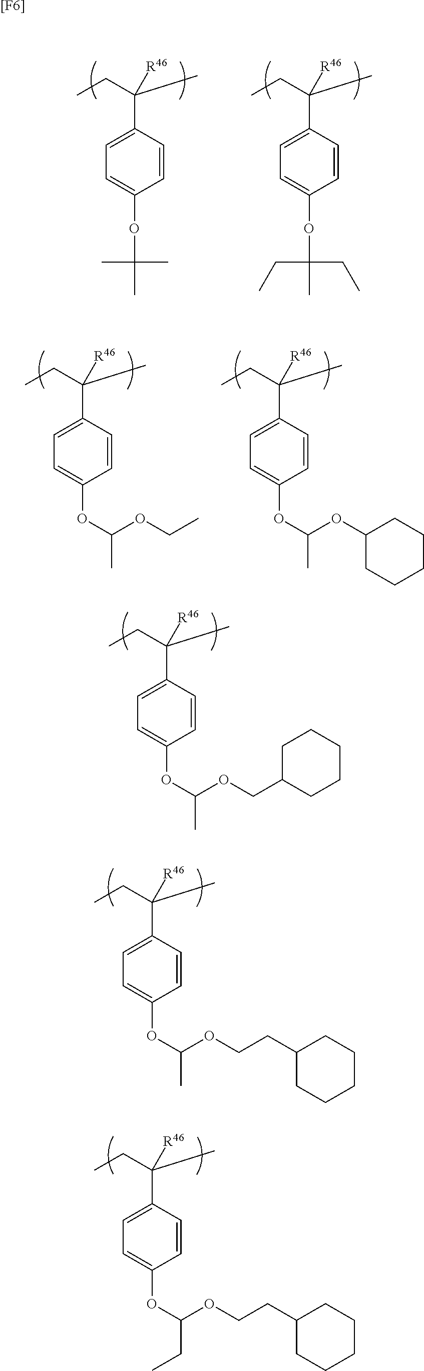

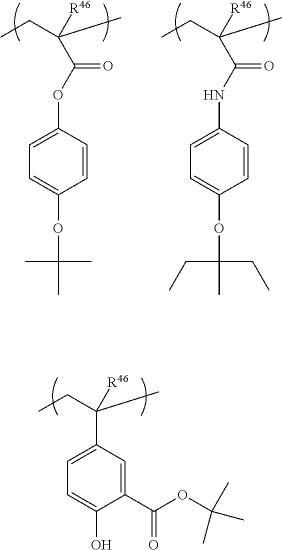

(In the above formulas, R⁴⁶ represents a hydrogen atom, a fluorine atom, a methyl group, or a trifluoromethyl group.)

The relative amount of structural unit (I) in all the structural units forming the polymer [A] is preferably 20 mol % or more, more preferably 25 mol % or more, still more preferably 30 mol % or more. Also, the relative amount of structural unit (I) in all the structural units forming the polymer [A] is preferably 80 mol % or less, more preferably 75 mol % or less, still more preferably 70 mol % or less. Adjusting the structural unit (I) content to satisfy the aforementioned conditions is preferred, since considerable difference in dissolution rate with respect to a developer between the light-exposed part and the light-unexposed part can be sufficiently attained, whereby favorable resist film pattern can be provided.

### [Additional Structural Unit]

Examples of the additional structural unit which the polymer [A] may have include the following structural units (II) to (IV).

### Structural Unit (II)

Preferably, the polymer [A] further includes a structural unit which has a hydroxy group bonded to an aromatic ring (except for the case corresponding to the structural units (I)) (hereinafter may also be referred to as a “structural unit (II)”). Incorporation of the structural unit (II) into the polymer [A] is preferred, since lithographic characteristics of the present composition (e.g., LWR performance and CDU (critical dimension uniformity) performance) can be further improved, and dissolution of the light-unexposed part of the radiation-sensitive composition containing any of the following compounds (b) in a developer can be effectively suppressed, whereby development failure can be satisfactorily reduced.

In the structural unit (II), examples of the aromatic ring to which a hydroxy group is bound include a benzene ring, a naphthalene ring, and an anthracene ring. Of these, a benzene ring and a naphthalene ring are preferred, with a benzene ring being more preferred. No particular limitation is imposed on the number and position of the hydroxy group(s) bound to the aromatic ring in the structural unit (II). The number of the hydroxy group(s) bound to the aromatic ring is preferably 1 to 3, more preferably 1 or 2. Examples of the structural unit (II) include the structural units represented by the following formula (ii):

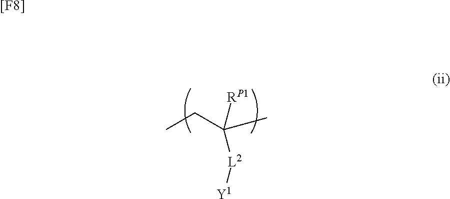

(in formula (ii), R^{P1} represents a hydrogen atom, a fluorine atom, a methyl group, or a trifluoromethyl group; L² represents a single bond, —O—, —CO—, —COO—, or —CONH—; and Y¹ represents a monovalent group having a hydroxy group bonded to an aromatic ring).

In the above formula (ii), R^{P1} is preferably a hydrogen atom or a methyl group, and L² is preferably a single bond or —COO—, from the viewpoint of co-polymerizability of a monomer to form the structural unit (II).

Specific examples of the structural unit (II) include the structural units represented by the following formulas:

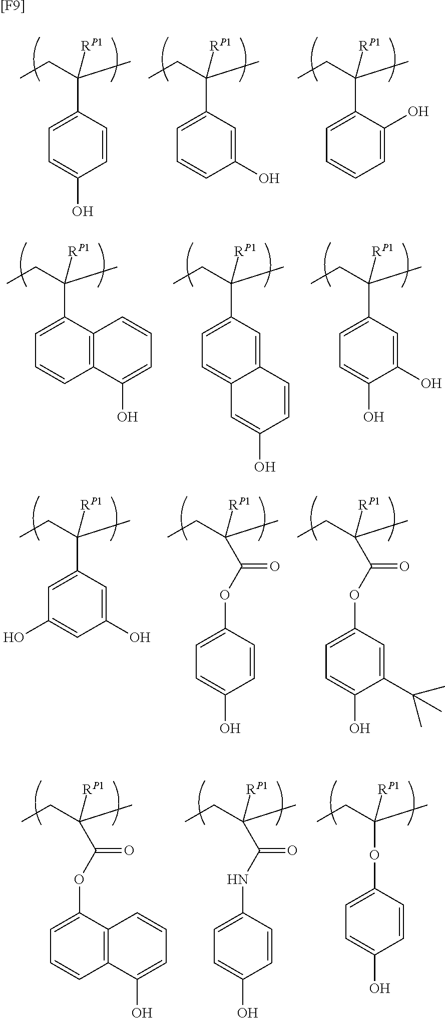

(in formulas, R^{P1} represents a hydrogen atom, a fluorine atom, a methyl group, or a trifluoromethyl group).

In the polymer [A], the relative amount of structural unit (II) in all the structural units forming the polymer [A] is preferably 5 mol % or more, more preferably 10 mol % or more, still more preferably 15 mol % or more. Also, the relative amount of structural unit (II) in all the structural units forming the polymer [A] is preferably 90 mol % or less, more preferably 80 mol % or less, still more preferably 60 mol % or less. By adjusting the structural unit (II) content to satisfy the above conditions, lithographic characteristics of the present composition can be further enhanced, which is preferred.

In the case where a polymer including the structural unit (II) is produced as the polymer [A], the structural unit (II) may be prepared by conducting polymerization while a phenolic hydroxy group is protected by a protective group such as an alkali-releasable group during polymerization, and then conducting deprotection through hydrolysis Notably, the polymer [A] may include a structural unit in which an acid-releasable group and a hydroxy group are bound to aromatic rings which are identical to or different from one another. As used herein, the structural unit in which an acid-releasable group and a hydroxy group are bound to aromatic rings which are identical to or different from one another is also classified to the structural unit (I).

### Structural Unit (III)

The polymer [A] may further includes a structural unit having a ring structure formed from a lactone structure, a cyclic carbonate structure, a sultone structure, or a combination of two or more members thereof (hereinafter may also be referred to as a “structural unit (III)”). Incorporation of the structural unit (III) into the polymer [A] is preferred, since solubility of the composition in a developer can be controlled, whereby lithographic characteristics of the present composition can be further enhanced. Also, the presence of the structural unit (III) in polymer [A] can improve close adhesion between a substrate and a resist film formed from the present composition.

Examples of the structural unit (III) includes the structural units represented by the following formulas:

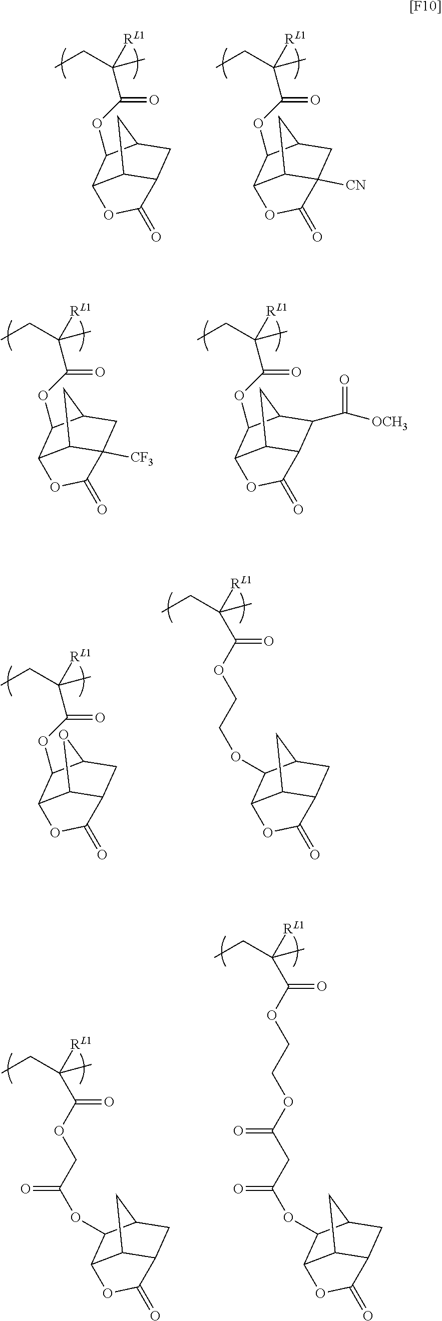

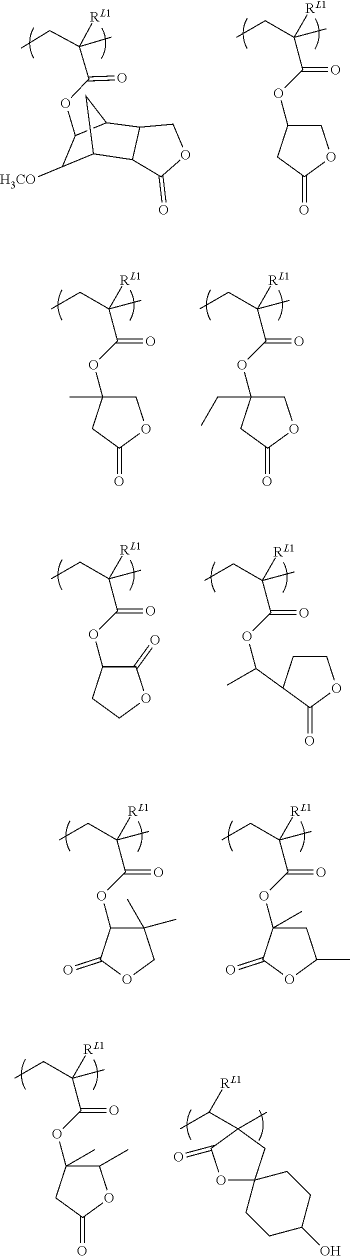

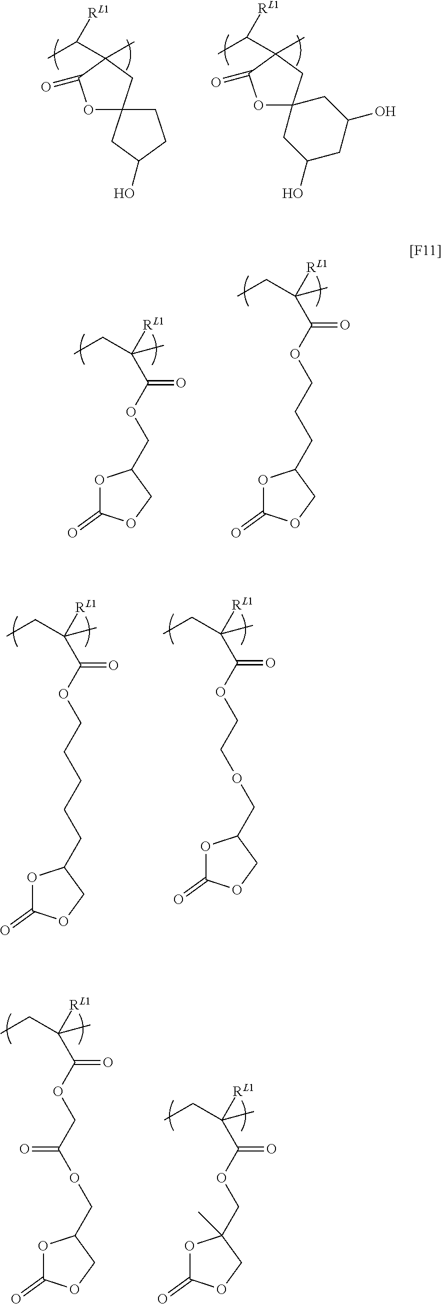

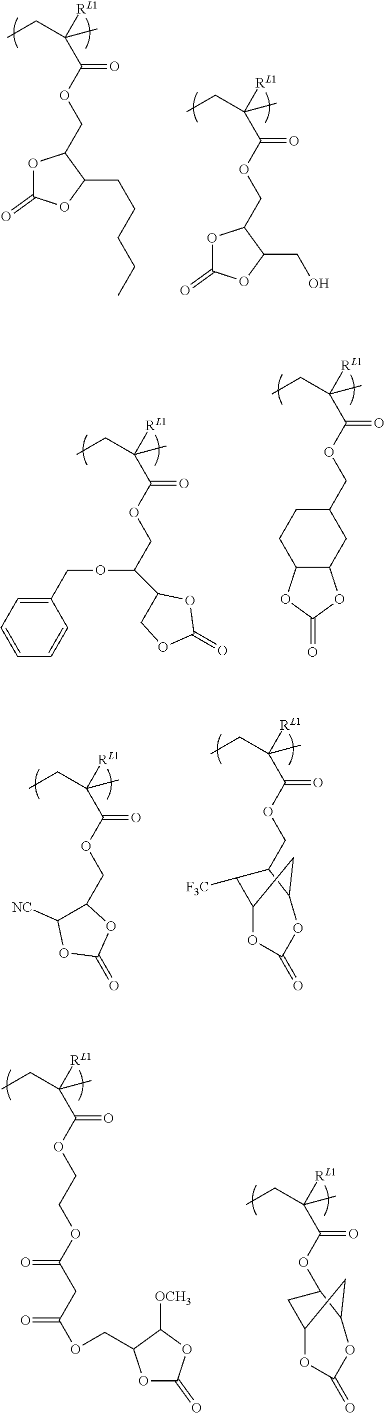

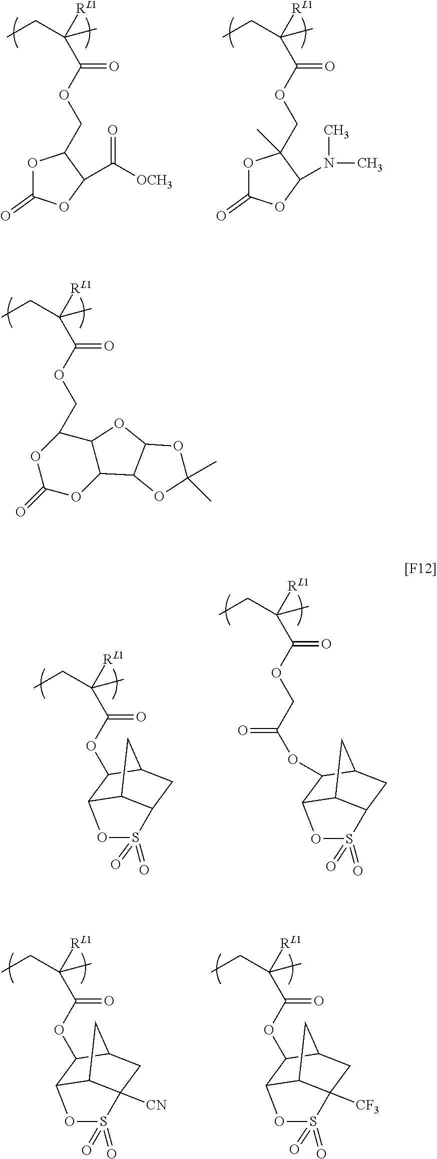

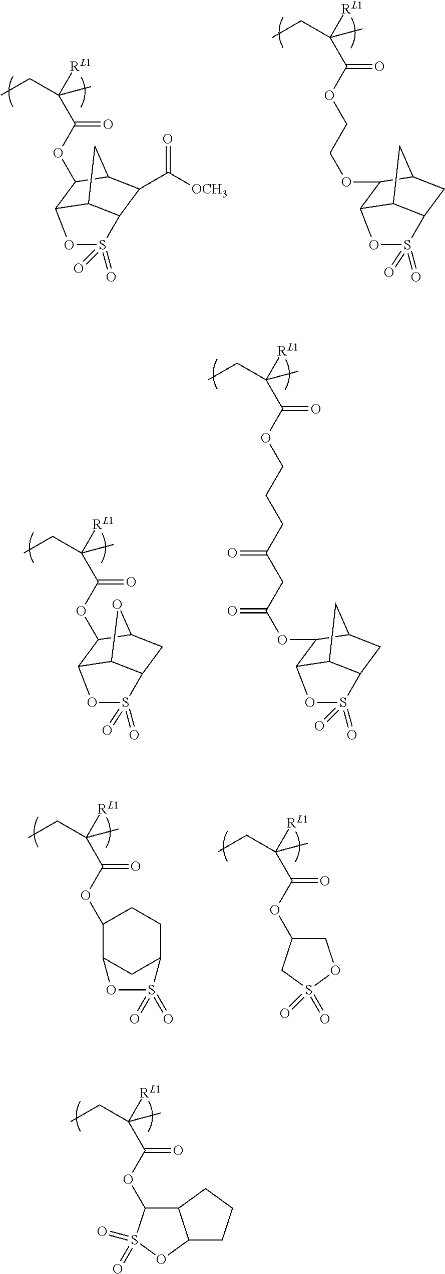

(in formulas, R^{L1} represents a hydrogen atom, a fluorine atom, a methyl group, or a trifluoromethyl group).

When the polymer [A] includes the structural unit (III), the relative amount of structural unit (III) in all the structural units forming the polymer [A] is preferably 1 mol % or more, more preferably 3 mol % or more, still more preferably 5 mol % or more. Also, the relative amount of structural unit (III) in all the structural units forming the polymer [A] is preferably 50 mol % or less, more preferably 30 mol % or less, still more preferably 15 mol % or less. Adjusting of the structural unit (III) content to satisfy the above conditions is preferred, since the lithographic characteristics of the present composition can be further enhanced, and close adhesion between the resist film obtained from the present composition and a substrate can be further enhanced.

### Structural Unit (IV)

The polymer [A] may further include a structural unit having an alcoholic hydroxy group (except for a structural unit corresponding to the structural units (I) to (III)) (hereinafter may also be referred to as a “structural unit (IV)”). As used herein, the term “alcoholic hydroxy group” refers to a group having a structure in which a hydroxy group is directly bound to an aliphatic hydrocarbon group. The aliphatic hydrocarbon group may be a chain hydrocarbon group or an alicyclic hydrocarbon group. By incorporating the structural unit (IV) into the polymer [A], solubility of the composition in a developer can be improved, whereby lithographic characteristics of the present composition can be further improved, which is preferred.

The structural unit (IV) is preferably a structural unit derived from an unsaturated monomer having an alcoholic hydroxy group. No particular limitation is imposed on the unsaturated monomer, and examples thereof include 3-hydroxyadamantan-1-yl (meth)acrylate and 2-hydroxyethyl (meth)acrylate.

When the polymer [A] includes the structural unit (IV), the relative amount of structural unit (IV) in all the structural units forming the polymer [A] is preferably 1 mol % or more, more preferably 3 mol % or more. Also, the relative amount of structural unit (IV) in all the structural units forming the polymer [A] is preferably 30 mol % or less, more preferably 20 mol % or less.

In addition the aforementioned structural units, examples of the additional structural unit include the following structural units such as
a structural unit having a partial structure which can generate acid in the present composition (e.g., a structural unit having a partial structure formed of a triarylsulfonium cation and an organic anion or a structural unit having a partial structure formed of a diaryliodonium cation and an organic anion);
a structural unit having a cyano group, a nitro group, or a sulfonamide group (e.g., a structural unit derived from 2-cyanomethyladamantan-2-yl (meth)acrylate); and
a structural unit having a non-acid-releasable hydrocarbon group (e.g., a structural unit derived from styrene, a structural unit derived from vinylnaphthalene, or a structural unit derived from n-pentyl (meth)acrylate.

The relative amount of any of these structural units may be appropriately set in accordance with the type of each structural unit, so long as the effects of the present disclosure are not impaired.

In the present composition, the polymer [A] content, with respect to the entire amount of the solid contained in the present composition, is preferably 50 mass % or more, more preferably 55 mass % or more, still more preferably 60 mass % or more. Also, the polymer [A] content, with respect to the entire amount of the solid contained in the present composition, is preferably 99 mass % or less, more preferably 98 mass % or less, still more preferably 95 mass % or less. The polymer [A] generally forms a base resin of the present composition. As used herein, the term “base resin” refers to a polymer component accounting for 50 mass % or more of the entire amount of the solid contained in the present composition. The present composition may contain the polymer [A] singly or in combination of two or more species.

The weight average molecular weight (Mw) of the polymer [A], which is determined through gel permeation chromatography (GPC) and is reduced to polystyrene, is preferably 1,000 or more, more preferably 2,000 or more, still more preferably 3,000 or more, yet more preferably 4,000 or more. Also, the Mw of the polymer [A] is preferably 50,000 or less, more preferably 30,000 or less, still more preferably 20,000 or less, yet more preferably 15,000 or less. Adjusting the Mw of the polymer [A] so as to satisfy the above conditions is preferred, since coatability of the present composition can be improved, and development failure can be sufficiently suppressed.

The ratio (Mw/Mn) of Mw to the number average molecular weight (Mn) of the polymer [A], which is determined through gel permeation chromatography (GPC) and is reduced to polystyrene, is preferably 5.0 or less, more preferably 3.0 or less, still more preferably 2.0 or less, yet more preferably 1.8 or less. Also, the Mw/Mn of the polymer [A] is generally 1 or more, preferably 1.3 or more. The polymer [A] can be synthesized through, for example, polymerizing monomers for providing corresponding structural units in the presence of a known radical polymerization initiator or the like in an appropriate solvent.

### <Acid-Generator [B]>

The acid-generator [B] is a substance which can provide a component contained in the present composition with an acid through exposure to light. Typically, the acid-generator [B] has a structure derived from an onium salt between a radiation-sensitive onium cation and an organic anion (i.e., a conjugate base of the corresponding acid). The organic anion is generally an anion formed by removing a proton from the acid residue of the organic acid. Through action of radiation, the acid-generator [B] releases an organic anion via decomposition of the radiation-sensitive onium cation, and the thus-released organic anion bonds to hydrogen extracted from a component contained in the present composition (e.g., the acid-generator [B] itself or a solvent), whereby a component in the present composition is provided with an acid.

The acid-generator [B] encompasses an acid-generator which can generate a strong acid (e.g., sulfonic acid, imidic acid, or methide acid) by light exposure and a light-degradable base, which is an acid diffusion-suppressing agent incorporated into a composition for suppressing diffusion of an acid to a light-unexposed part. As uses herein, the term “radiation” encompasses to an electron beam (e.g., visible light, UV ray, far-UV ray, or extreme ultraviolet ray (EUV)) and an electromagnetic wave (e.g., X-ray or γ-ray).

The present composition contains, as the acid-generator [B], contains at least one compound (b) selected from the group consisting of the compounds represented by the aforementioned formula (1) and the compounds represented by the aforementioned formula (2).

[Compound (b)]

The compound (b) is a compound including a cation and a sulfonamide-type organic anion having a partial structure in which an iodine atom is bound to a carbon atom forming an aromatic ring or a carbon-carbon double bond. Preferably, the compound (b) is incorporated into the present composition as a light-degradable base which generates an acid (sulfonamide) derived from an organic anion included in the compound (b) upon irradiation with a radiation. When the present composition contains a light-degradable base and an acid-generator, the light-degradable base serves as a component which generates an acid weaker than the acid-generator in the present composition upon light exposure. The degree of acidity can be evaluated on the basis of acid dissociation constant (pKa). The acid dissociation constant (pKa) of the acid generated in the present composition by the action of the light-degradable base is preferably −3 or higher, more preferably −1 to 7, still more preferably 0 to 5.

### Anion in the Aforementioned Formula (1)

In the aforementioned formula (1), the C1 to C20 monovalent organic group represented by R¹ is preferably a C1 to C20 monovalent hydrocarbon group or a C1 to C20 hydrocarbon group in which a hydrogen atom is substituted with a substituent. Examples of the C1 to C20 monovalent hydrocarbon group include those exemplified in relation to the C1 to C20 monovalent hydrocarbon groups represented by any of R⁴³ to R⁴⁵ and R⁴⁷ to R⁴⁹ in the aforementioned formulas (i-1) and (i-2). Examples of the substituent include a fluorine atom, a chlorine atom, a bromine atom, an iodine atom, a hydroxy group, a carboxy group, a cyano group, a nitro group, an amino group, an alkoxy group, alkoxycarbonyl group, an alkoxycarbonyloxy group, an aryloxy group, an aryloxycarbonyl group, an aryloxycarbonyloxy group, an acyl group an acyloxy group, —OSO₂—R^{k}, —SO₂—R^{k}, —OR^{k}, —O—CO—R^{k}, —O—R^{kk}—COOR^{k}, —R^{kk}—CO—R^{k}, and —S—R^{k}. R^{k} represents a C1 to C10 monovalent hydrocarbon group, and R^{kk} represents a single bond or a C1 to C10 divalent hydrocarbon group (the same definitions of R^{k} and R^{kk} applying to the following description).

R¹ preferably has a halogen atom, more preferably has one or both of a fluorine atom and an iodine atom. Specifically, R¹ preferably has a fluorine atom, from the viewpoints of establishing dissolution performance of a light-exposed part of a resist pattern formed by use of the present composition in a developer and reduction of development failures (e.g., breaking failure) caused by dissolution of a light-unexposed part, and enhancing in sensitivity of the present composition, R¹ preferably has an iodine atom, more preferably a partial structure in which an iodine atom is bound to an aromatic ring.

When R² is a C1 to C20 divalent group which is bound to N⁻ in the aforementioned formula (1) via —CR⁴R⁵—, R⁴ and R⁵ are a hydrogen atom, a C1 to C3 monovalent hydrocarbon group, or —COOR⁶. The C1 to C3 monovalent hydrocarbon group represented by R⁴ or R⁵ is preferably an alkyl group. Specific examples of the alkyl group include a methyl group, an ethyl group, an n-propyl group, and an isopropyl group. In a group represented by —COOR⁶, examples of the C1 to C6 monovalent hydrocarbon group represented by R⁶ include those exemplified in relation to the C1 to C6 monovalent hydrocarbon groups represented by any of R⁴³ to R⁴⁵ and R⁴⁷ to R⁴⁹ in the aforementioned formulas (i-1) and (i-2). R⁴ and R⁵ may be identical to or different from each other.

When R² is a C1 to C20 divalent group which is bound to N⁻ in the aforementioned formula (1) via —CR⁴R⁵—, specific examples of —CR⁴R⁵— include, but are not limited to, groups represented by the following formulas:

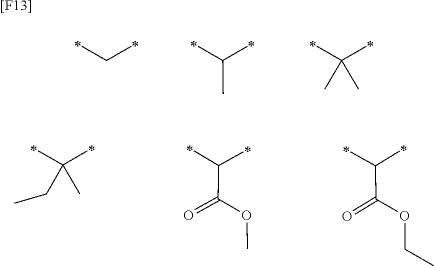

(in formulas, “*” represents a chemical bond).

When R² is a C1 to C20 divalent group which is bound to N⁻ in the aforementioned formula (1) via an aromatic ring, the aromatic ring may be an aromatic hydrocarbon ring or an aromatic heterocycle. Specific examples of the aromatic ring bound to N⁻ include a benzene ring, a naphthalene ring, an anthracene ring, a phenanthrene ring, a tetracene ring, a pyrene ring, a triphenylene ring, a furan ring, a thiophene ring, a pyrrole ring, and a pyridine ring. Among them, the aromatic ring bound to N⁻ is preferably a benzene ring, a naphthalene ring, an anthracene ring, a phenanthrene ring, a pyrene ring, a furan ring, or a thiophene ring, with a benzene ring or a naphthalene ring being more preferred, a benzene ring being still more preferred. Notably, the aromatic ring bound to N⁻ may have a substituent. Examples of the substituent include a fluorine atom, a chlorine atom, a bromine atom, an iodine atom, a hydroxy group, a cyano group, a nitro group, a carboxy group, and a C1 to C4 alkoxy group.

When R² is a C1 to C20 divalent group which is bound to N⁻ in the aforementioned formula (1) via an aromatic ring, specific examples of the aromatic ring (a divalent aromatic ring group) bound to N⁻ include a group formed by removing two hydrogen atoms from the ring moiety of any of the aromatic hydrocarbon rings or aromatic heterocycles exemplified above. Examples thereof include, but are not limited to, groups represented by the following formulas:

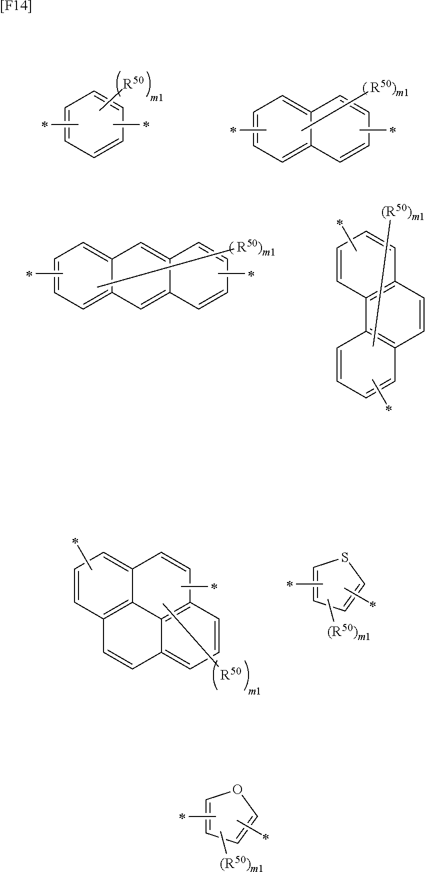

(in formulas, R⁵⁰ represents a fluorine atom, a chlorine atom, a bromine atom, an iodine atom, a hydroxy group, a cyano group, a nitro group, or a C1 to C4 alkoxy group; ml is an integer of 0 to 2; and “*” represents a chemical bond).

From the viewpoint of enhancing basicity and hydrophobicity of the compounds represented by the aforementioned formula (1) to thereby improve a development failure reducing effect, R² is preferably, among others, a single bond or a divalent group represented by the following formula (3).

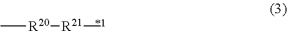

(In formula (3), R²⁰ represents a single bond or a divalent linking group; R²¹ represents —CR⁴R⁵— or a divalent aromatic ring group; R⁴ and R⁵ have the same definitions as those of the aforementioned formula (1); and “*1” represents a chemical bond bound to N⁻ in the aforementioned formula (1).)

In the aforementioned formula (3), when the group represented by R²⁰ is a divalent linking group, examples of the linking group include a hetero atom-containing group U¹ such as a carbonyl group, an ether group, a carbonyloxy group, a sulfide group, a thiocarbonyl group, a sulfonyl group, an amide group, or a carbonate group;
a cyclic group U² formed by removing two hydrogen atoms from a hetero atom-containing group such as a cyclic ether, a dioxalene ring, a dioxane ring, a sultone ring, a lactam ring, or a lactone ring;
a divalent hydrocarbon group such as an alkanediyl group, an alkenediyl group, or a cycloalkanediyl group; and
a divalent group formed by substituting any methylene group of a C2 to C10 alkanediyl group or alkenediyl group with the aforementioned hetero atom-containing group U¹ or cyclic group U². Specific examples of the —CR⁴R⁵— and the divalent aromatic ring group represented by R²¹ are the same as mentioned above.

From the viewpoint of ease of synthesis of the compounds represented by the aforementioned formula (1), R² is preferably, among others, a single bond or a C1 to C20 divalent group which is bound to N⁻ via —CR⁴R⁵— or a benzene ring, more preferably a single bond or a C1 to C10 divalent group which is bound to N⁻ via —CR⁴R⁵—, still more preferably a single bond.

A¹ is a group formed by removing (m+n+1) hydrogen atoms from an aromatic ring. Examples of the aromatic ring include a benzene ring, a naphthalene ring, an anthracene ring, and a phenanthrene ring. Among them, the aromatic ring included in A¹ is preferably a benzene ring or a naphthalene ring, more preferably a benzene ring. Examples of the substituent represented by R³ include the same groups as exemplified as the substituent which R¹ may have (except for an iodine atom).

From the viewpoint of improving the sensitivity and LWR performance of the present composition, n is preferably 1 to 5, more preferably 1 to 4, still more preferably 1 to 3, and m is preferably 0 to 3, more preferably 0 to 2.

Specific examples of the anion of the compound represented by the aforementioned formula (1) (hereinafter may also be referred to as a “compound (b-1)”) include anions each represented by the following formulas (b-1-1) to (b-1-28).

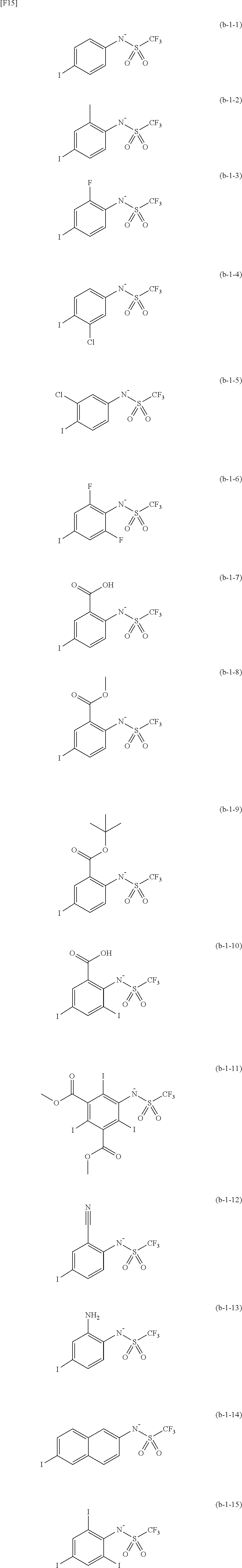

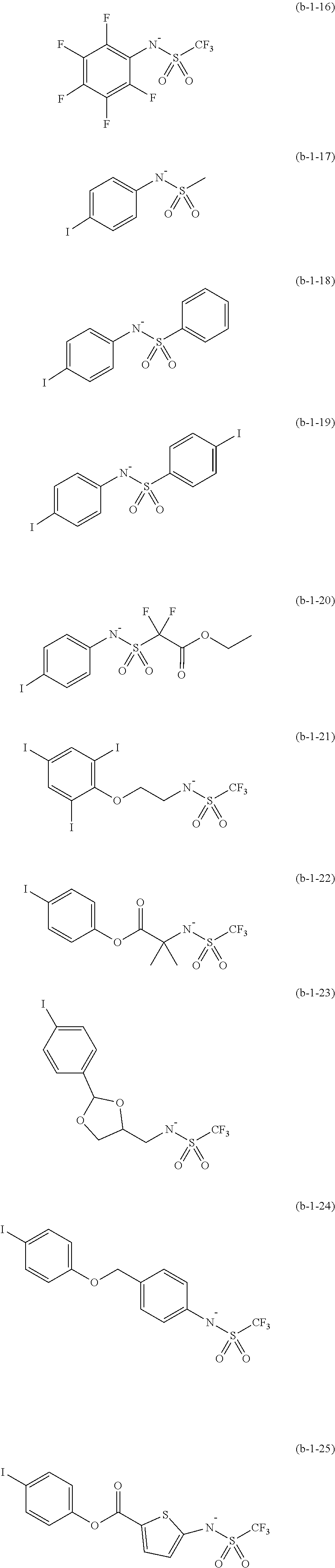

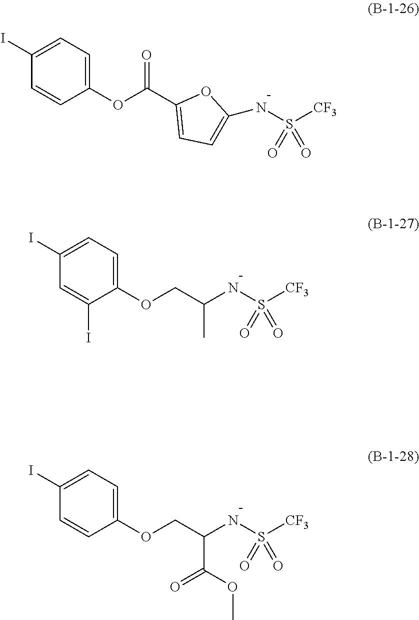 [SMILES: O=C(Oc1ccc(I)cc1)c1ccc(NS(=O)(=O)C(F)(F)F)o1 ; CC(COc1ccc(I)cc1I)NS(=O)(=O)C(F)(F)F ; COC(=O)C(COc1ccc(I)cc1)NS(=O)(=O)C(F)(F)F]

### Anion in the Aforementioned Formula (2)

In the aforementioned formula (2), when R⁷ is a divalent group represented by the aforementioned formula (r-1), specific examples of the C1 to C10 (t+2)-valent hydrocarbon group represented by R⁹ include groups formed by further removing (t+1) hydrogen atoms from any of the groups exemplified as the C1 to C10 monovalent hydrocarbon group, among the monovalent hydrocarbon groups represented by R⁴³ to R⁴⁵ and R⁴⁷ to R⁴⁹ in the aforementioned formulas (i-1) and (i-2). R¹⁵ is preferably a hydrogen atom or a C₁ to C₅ alkyl group. When a group represented by R⁹ has a substituent, examples of the substituent include a fluorine atom, a chlorine atom, a bromine atom, a hydroxy group, a carboxy group, a cyano group, a nitro group, and an amino group.

R⁹ is preferably, among others, a C1 to C10 (t+2)-valent chain hydrocarbon group; a C1 to C10 (t+2)-valent organic group formed by substituting any methylene group of a chain hydrocarbon group with —O—, —S—, —NR¹⁵—, or a carbonyl group; or a (t+2)-valent group formed by substituting any hydrogen atom of a chain hydrocarbon group or a (t+2)-valent organic group with a substituent.

A² is a group formed by removing (p+q+1) hydrogen atoms from an aromatic ring. Examples of the aromatic ring include a benzene ring, a naphthalene ring, an anthracene ring, and a phenanthrene ring. Among them, the aromatic ring included in A² is preferably a benzene ring or a naphthalene ring, particularly preferably a benzene ring. Examples of the substituent represented by R¹⁰ include the same groups as exemplified as the substituent which R¹ in the aforementioned formula (1) may have (except for an iodine atom).

From the viewpoint of improving the sensitivity and LWR performance of the present composition, q is preferably 1 to 5, more preferably 1 to 4, still more preferably 1 to 3, and p is preferably 0 to 3, more preferably 0 to 2.

The group represented by the aforementioned formula (r-1) is preferably, among others, a group represented by the following formula (r-1A).

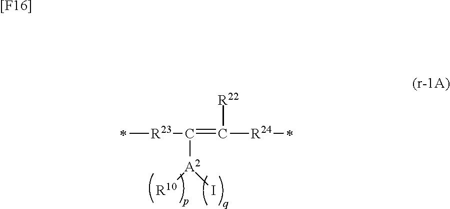

(In formula (r-1A), R²² represents a hydrogen atom, an iodine atom, or a monovalent organic group; each of R²³ and R²⁴ independently represents a single bond, —O—, —S—, —NR²⁶—, a carbonyl group, a C1 to C3 divalent chain hydrocarbon group, or a C1 to C6 divalent group formed by substituting any methylene group of a chain hydrocarbon group with —O—, —S—, —NR²⁶—, or a carbonyl group; R²⁶ represents a hydrogen atom or a C₁ to C₅ monovalent hydrocarbon group; A², R¹⁰, p, and q have the same definitions as those of A², R¹⁰, p, and q in the aforementioned formula (r-1); and “*” represents a chemical bond.)

In the aforementioned formula (r-1A), examples of the monovalent organic group represented by R²² include the groups as exemplified in relation to monovalent organic groups represented by R¹ in the aforementioned formula (1). Examples of the C₁ to C₅ monovalent hydrocarbon group represented by R²⁶ include the same C1 to C5 groups, among the groups exemplified as the C1 to C20 monovalent hydrocarbon groups represented by R⁴³ to R⁴⁵ and R⁴⁷ to R⁴⁹ in the aforementioned formulas (i-1) and (i-2). R²⁶ is preferably a hydrogen atom or a C₁ to C₃ alkyl group.

One of R²³ and R²⁴ is bound to the sulfonyl group in the aforementioned formula (2), and the other is bound to the carbonyl group in the aforementioned formula (2). From ease of synthesis of the compound represented by the aforementioned formula (2), the group among R²³ and R²⁴ which is bound to the sulfonyl group in the aforementioned formula (2) is preferably —O—. Also, the group which is bound to the carbonyl group in the aforementioned formula (2) is preferably a single bond or a C₁ to C₃ alkanediyl group, more preferably a single bond.

When the R⁷ in the aforementioned formula (2) is a divalent group represented by the aforementioned formula (r-2), A³ is a group formed by removing (r+s+2) hydrogen atoms from an aromatic ring. Examples of the aromatic ring include a benzene ring, a naphthalene ring, an anthracene ring, and a phenanthrene ring. Among them, the aromatic ring included in A³ is preferably a benzene ring or a naphthalene ring, particularly preferably a benzene ring. Examples of the substituent of R¹¹ include the same groups as exemplified as the substituent which R¹ in the aforementioned formula (1) may have (except for an iodine atom).

In R¹⁷ and R¹⁸, the group bound to a sulfonyl group in the aforementioned formula (2) is preferably —O—, for the same reasons in relation to R²³ and R²⁴. Also, the group bound to a carbonyl group in the aforementioned formula (2) is preferably a single bond or a C1 to C3 alkanediyl group, more preferably a single bond.

From the viewpoint of suppressing development failure of the resist pattern formed by use of the present composition, the “s” in the aforementioned formula (r-2) is preferably 1 to 5, more preferably 1 to 4, still more preferably 1 to 3, and r is preferably 0 to 3, more preferably 0 to 2.

When the R⁷ in the aforementioned formula (2) is a divalent group represented by the aforementioned formula (r-3), specific examples of the C1 to C5 monovalent hydrocarbon group represented by any of R¹² and R¹⁶ include the same groups as C1 to C5 groups among the examples of the C1 to C20 monovalent hydrocarbon groups represented by R⁴³ to R⁴⁵ and R⁴⁷ to R⁴⁹ in the aforementioned formulas (i-1) and (i-2). R¹² is preferably a hydrogen atom, an iodine atom, or a C1 to C5 alkyl group, more preferably a hydrogen atom, an iodine atom, or a C1 to C3 alkyl group.

In R¹³ and R¹⁴, the group bound to a sulfonyl group in the aforementioned formula (2) is preferably —O—, for the same reasons in relation to R²³ and R²⁴. Also, the group bound to a carbonyl group in the aforementioned formula (2) is preferably a single bond or a C1 to C3 alkanediyl group, more preferably a single bond.

Specific examples of the anion of the compound represented by the aforementioned formula (2) (hereinafter may also be referred to as a “compound (b-2)”) include anions each represented by the following formulas (b-2-1) to (b-2-11). Anions represented by the following formulas (b-2-1) to (b-2-5) are examples of the anion represented by the aforementioned formula (2) in which R⁷ is a group represented by the aforementioned formula (r-1). Anions represented by the following formulas (b-2-6) to (b-2-8) are examples of the anion represented by the aforementioned formula (2) in which R⁷ is a group represented by the aforementioned formula (r-2). Anions represented by the following formulas (b-2-9) to (b-2-11) are examples of the anion represented by the aforementioned formula (2) in which R⁷ is a group represented by the aforementioned formula (r-3).

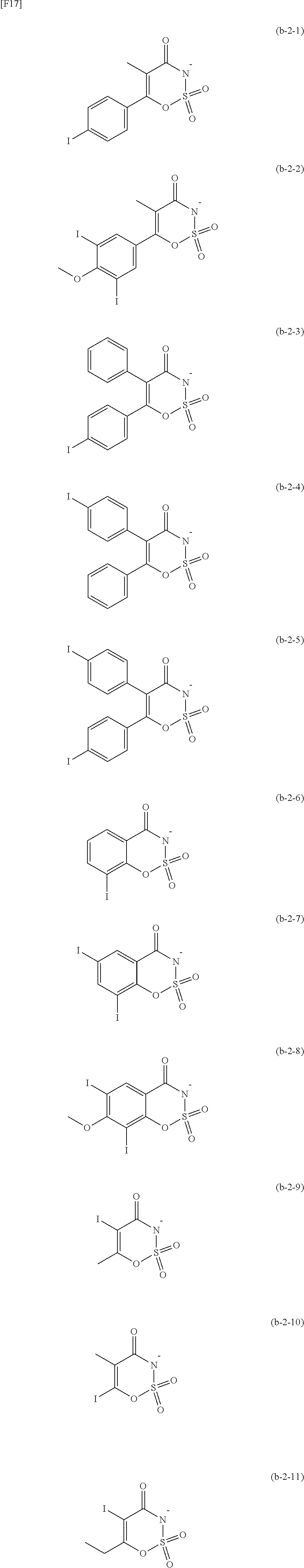

### Cations in Formulas (1) and (2)

M^{a+} in the aforementioned formula (1) and M^{b+} in the aforementioned formula (2) are preferably an organic cation, particularly preferably a radiation-sensitive onium cation.

No particular limitation is imposed on the structures of M^{a+} and M^{b+}. From the viewpoint of achieving favorable lithographic characteristics of the present composition, M^{a+} and M^{b+} are preferably a sulfonium cation, an iodonium cation, or an ammonium cation, more preferably a triarylsulfonium cation or a diaryliodonium cation, still more preferably a triarylsulfonium cation or a diaryliodonium cation, having an aryl group in which at least one hydrogen atom is substituted with an iodine atom.

In the case where “a” in the aforementioned formula (1) and “b” in the aforementioned formula (2) each are 1, specific examples of M^{a+} and M^{b+} include a cation represented by the following formula (4), a cation represented by the following formula (5), a cation represented by the following formula (6), and a cation represented by the following formula (7).

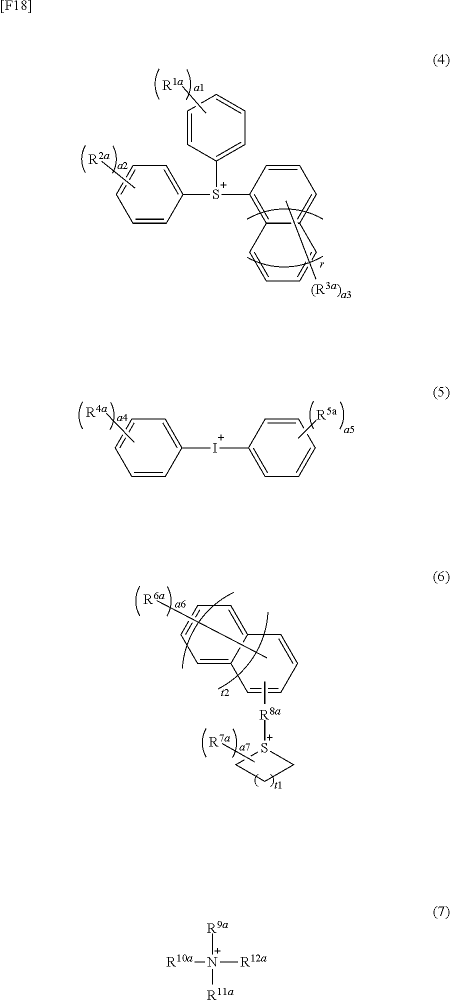

(In formula (4), each of R^{1a} and R^{2a} independently represents a monovalent substituent, or a single bond or a divalent group linked to a ring formed by combining R^{1a} and R^{2a}; R^{3a} represents a monovalent substituent; each of a1 and a2 is an integer of 0 to 5; a3 is an integer of 0 to (2×r+5); and r is 0 or 1;
in formula (5), each of R^{4a} and R^{5a} independently represents a monovalent substituent; and each of a4 and a5 is an integer of 0 to 5;
in formula (6), a6 is an integer of 0 to 7; when a6 is 1, R^{6a} is a C1 to C20 monovalent organic group, a hydroxy group, a nitro group, or a halogen atom; when a6 is 2 or greater, a plurality of groups “R^{6a}”, which are identical to or different from one another, represent a C1 to C20 monovalent organic group, a hydroxy group, a nitro group, or a halogen atom, or a 4- to 20-membered ring structure formed with a carbon atom to which a linked moiety formed of two or more of the plurality of groups “R^{6a}” are bonded; a7 is an integer of 0 to 6; when a7 is 1, R^{7a} is a C1 to C20 monovalent organic group, a hydroxy group, a nitro group, or a halogen group; when a7 is 2 or greater, a plurality of groups “R^{7a}”, which are identical to or different from one another, represent a C1 to C20 monovalent organic group, a hydroxy group, a nitro group, or a halogen atom, or a 3- to 20-membered ring structure formed with a carbon atom to which a linked moiety formed of two or more of the plurality of groups “R^{7a}” are bonded; t1 is an integer of 0 to 3; R^{8a} represents a single bond or a C1 to C20 divalent organic group; and t2 is 0 or 1; and
in formula (7), each of R^{9a} and R^{10a} independently represents a hydrogen atom or a monovalent organic group, or a ring structure formed with a nitrogen atom to which a linked moiety formed of R^{9a} and R^{10a} is bonded; and each of R^{11a} and R^{12a} independently represents a hydrogen atom or a monovalent organic group, or a ring structure formed with a nitrogen atom to which a linked moiety formed of R^{11a} and R^{12a} is bonded.)

In the aforementioned formulas (4) and (5), examples of the monovalent substituent represented by any of R^{1a}, R^{2a}, R^{3a}, R^{4a}, and R^{5a} (hereinafter denoted by “R^{1a} to R^{5a}”) include a fluorine atom, a chlorine atom, a bromine atom, an iodine atom, a substituted or unsubstituted alkyl group, a substituted or unsubstituted alkoxy group, a substituted or unsubstituted cycloalkyl group, a substituted or unsubstituted cycloalkyloxy group, an ester group, an alkylsulfonyl group, a cycloalkylsulfonyl group, a hydroxy group, a carboxy group, a cyano group, and a nitro group.

The alkyl group represented by any of R^{1a} to R^{5a} may be linear-chain or branched. The alkyl group is preferably a C1 to C10, and examples thereof include methyl, ethyl, n-propyl, i-propyl, n-butyl, i-butyl, sec-butyl, t-butyl, n-pentyl, and neopentyl. Of these, the alkyl group represented by any of R^{1a} to R^{5a} is preferably a C1 to C5 alkyl group, with methyl, ethyl, n-butyl, and t-butyl being more preferred.

When any of R^{1a} to R^{5a} is an alkoxy group, specific examples thereof include a group in which any of the above-exemplified alkyl groups is attached to the alkyl moiety forming the alkoxy group. The alkoxy group is particularly preferably methoxy, ethoxy, n-propoxy, or n-butoxy.

The cycloalkyl group represented by any of R^{1a} to R^{5a} may be monocyclic or polycyclic. Examples of the monocyclic cycloalkyl group include cyclopropyl, cyclobutyl, cyclopentyl, cyclohexyl, and cyclooctyl. Examples of the polycyclic cycloalkyl group include norbornyl, adamantyl, tricyclodecyl, and tetracyclododecyl.

When any of R^{1a} to R^{5a} is a cycloalkyloxy group, specific examples thereof include a group in which any of the above-exemplified cycloalkyl groups is attached to the cycloalkyl moiety forming the cycloalkyloxy group. The alkoxy group is particularly preferably cyclopentyloxy or cyclohexyloxy.

When the group represented by any of R^{1a} to R^{5a} has a substituent, examples of the substituent include a fluorine atom, a chlorine atom, a bromine atom, an iodine atom, a hydroxy group, a carboxy group, a cyano group, a nitro group, and a C1 to C5 alkoxy group.

When any of R^{1a} to R^{5a} is an ester group (—COOR), specific examples of the hydrocarbon moiety (R) of the ester group include the above-exemplified, substituted or unsubstituted alkyl groups and substituted or unsubstituted cycloalkyl groups. When any of R^{1a} to R^{5a} is an ester group, R^{1a} to R^{5a} are preferably methoxycarbonyl, ethoxycarbonyl, or n-butoxycarbonyl.

When any of R^{1a} to R^{5a} is an alkylsulfonyl group, examples of the alkyl moiety forming the alkylsulfonium group include the above-exemplified substituted or unsubstituted alkyl groups. When any of R^{1a} to R^{5a} is a cycloalkylsulfonyl group, examples of the alkyl moiety forming the cycloalkylsulfonium group include the aforementioned, substituted or unsubstituted cycloalkyl groups.

When R^{1a} and R^{2a} represent a divalent group linked to a ring formed by combining R^{1a} and R^{2a}, R^{1a} and R^{2a} preferably form a single bond, —O—, —S—, —CO—, or —SO—.

In formula (6), each of R^{6a} and R^{7a} is preferably a substituted or unsubstituted C1 to C20 monovalent hydrocarbon group, —OR^{k}, —COOR^{k}, —O—CO—R^{k}, —O—R^{kk}—COOR^{k}, or —R^{kk}—CO—R^{k}. Examples of the C1 to C20 monovalent hydrocarbon group represented by R^{6a} or R^{7a} include the same groups as exemplified in relation to C1 to C20 monovalent hydrocarbon groups represented by R⁴³ to R⁴⁵ and R⁴⁷ to R⁴⁹ in the aforementioned formulas (i-1) and (i-2). In R^{6a} and R^{7a}, examples of the substituent which replaces a hydrogen atom of a hydrocarbon group include the same groups as exemplified in relation to substituents included in the aforementioned R^{1a} to R^{5a}.

Examples of the divalent organic group represented by R^{8a} include a group formed by removing one hydrogen atom from a C1 to C20 monovalent organic group exemplified in relation to R^{6a} and R^{7a}.

Each of R^{6a} and R^{7a} is preferably, among the aforementioned groups, an unsubstituted, linear-chain or branched monovalent alkyl group, a monovalent fluoroalkyl group, an unsubstituted monovalent aromatic hydrocarbon group, —OSO₂—R^{k}, or —SO₂—R^{k}. The number a6 is preferably an integer of 0 to 2, more preferably 0 or 1, still more preferably 0. The number a7 is preferably an integer of 0 to 2, more preferably 0 or 1, still more preferably 0. The number t2 is preferably 0, and the number t1 is preferably 2 or 3, more preferably 2.

In the aforementioned formula (7), examples of the monovalent organic group represented by any of R^{9a} to R^{12a} include the same groups as exemplified in relation to the monovalent organic group represented by R¹ in the aforementioned formula (1).

When a in the aforementioned formula (1) and b in the aforementioned formula (2) are 1, each of M^{a+} and M^{b+} is preferably a sulfonium cation or an iodonium cation, more preferably a cation represented by the aforementioned formula (4) or (6), still more preferably a cation represented by the aforementioned formula (4). When a in the aforementioned formula (1) and b in the aforementioned formula (2) are 2, each of M^{a+} and M^{b+} is preferably a sulfonium cation.

Specific examples of M^{a+} and M^{b+} include, but are not limited to, cations represented by the following formulas.

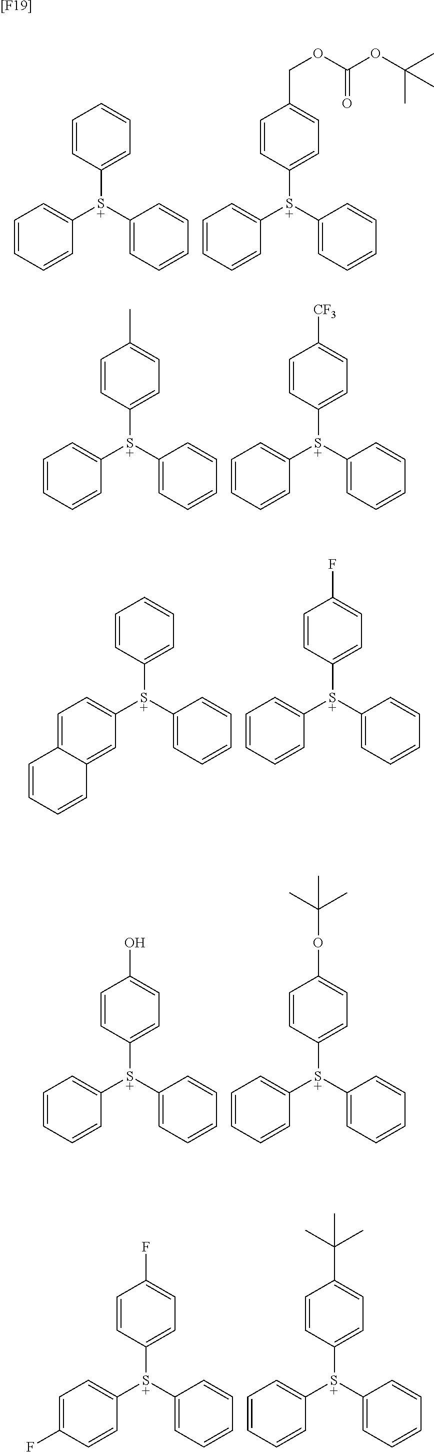

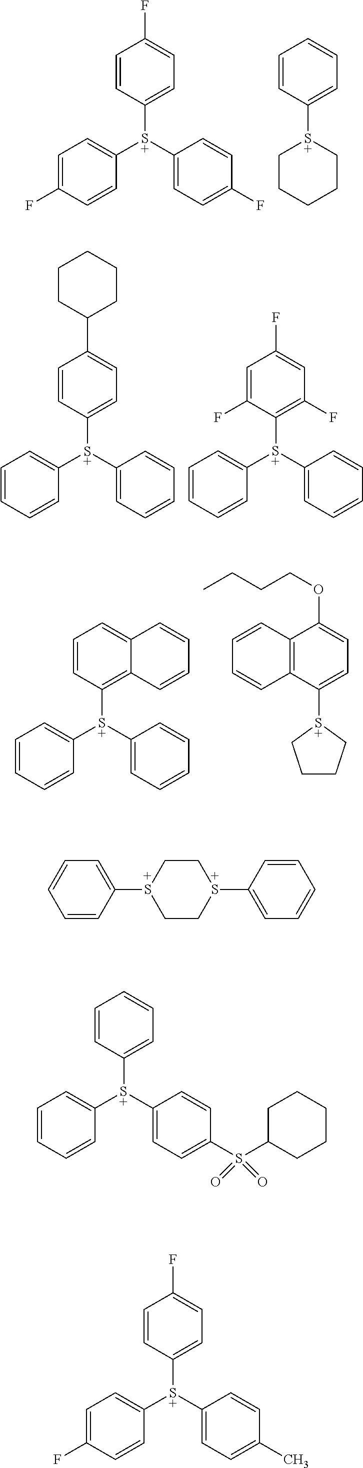

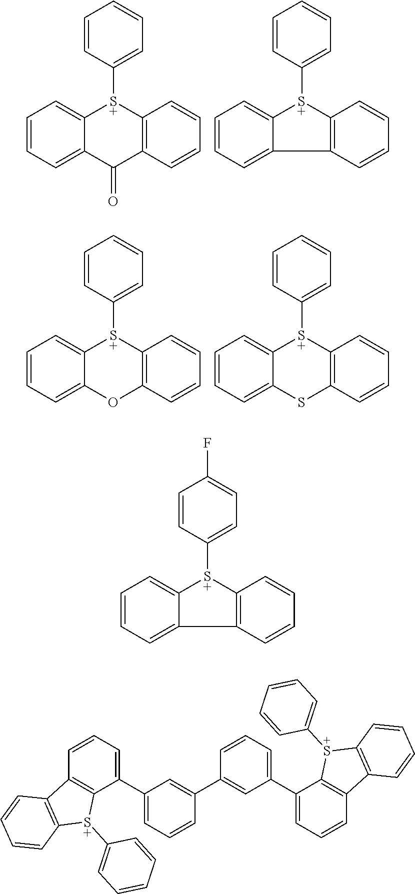

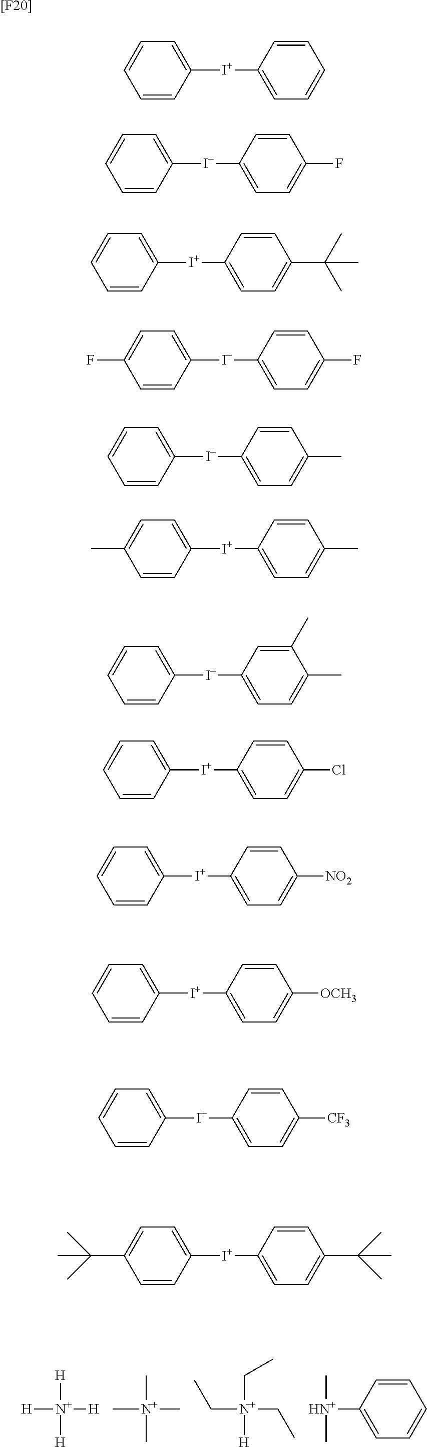

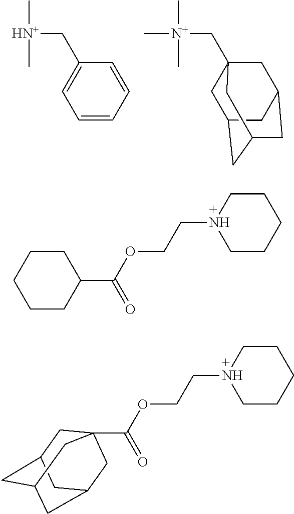

In the present composition, the relative amount of the compound (b) with respect to 100 parts by mass of the polymer [A] is preferably 0.001 parts by mass or more, more preferably 0.005 parts by mass or more, still more preferably 0.01 parts by mass or more. Also, the relative amount of the compound (b) with respect to 100 parts by mass of the polymer [A] is preferably 20 parts by mass or less, more preferably 10 parts by mass or less. Controlling the compound (b) content to satisfy the above conditions is preferred, since the effects of improving the sensitivity of the present composition and LWR performance and reducing development failure can be achieved in a well-balanced manner. The compound (b) may be used singly or in combination of two or more species.

<Synthesis of Compound (b)>

The compound (b) may be synthesized through customary methods of organic chemistry in appropriate combinations. In one procedure, compound (b-1) may be synthesized by reacting an amine compound having a partial structure represented by “(I)ₙ-A¹(R³)m-R²—” (e.g., 4-iodoaniline in synthesis of a compound of the aforementioned formula (1) (m=0, n=1, A¹=benzene ring, and R²=single bond)) with a sulfonic anhydride having group R¹ (e.g., trifluoromethanesulfonic anhydride) in an appropriate solvent optionally in the presence of a catalyst, and reacting the formed intermediate with a sulfonium chloride or the like which can impart an onium cation moiety thereto. Also, compound (b-2) may be synthesized by reacting a compound having a partial structure represented by R⁷ with a compound having group —SO₂—NCO— (e.g., chlorosulfonyl isocyanate) in an appropriate solvent optionally in the presence of a catalyst, and reacting the formed intermediate with a sulfonium chloride or the like which can impart an onium cation moiety thereto. However, the method of synthesizing the compound (b) is not limited to the above procedure.

The present composition may further contain, as the acid-generator [B], a compound differing from the compound (b) (hereinafter may also be referred to as an “additional acid-generator”). Examples of the additional acid-generator include a compound which can generate an acid stronger than the acid generated in the present composition by the compound (b) upon exposure of the present composition to light (hereinafter may also be referred to as an “acid-generating agent [C]”), and a compound which differs from the compound (b) and which can generate an acid weaker than the acid generated in the present composition by the acid-generating agent [C] upon exposure of the present composition to light (hereinafter may also be referred to as a “compound (b)”).

### <Acid-Generating Agent [C]>

The acid-generating agent [C] is typically an onium salt composed of an onium cation and an organic anion. Through bonding of an organic anion released from the acid-generating agent [C] upon light exposure to hydrogen, an acid is generated in the present composition. The thus-generated acid release an acid-releasable group in the polymer [A] to thereby form an acid radical, whereby solubility of the polymer [A] in a developer can be modified, which is effective for forming a suitable resist pattern. Also, through incorporation of the acid-generating agent [C] and the compound (b) into the present composition, the compound (b) functions as a quencher, to thereby suppress diffusion of the acid generated by the acid-generating agent [C], whereby a suitable resist pattern can be formed.

No particular limitation is imposed on the acid-generating agent [C] incorporated into the present composition, and any known acid-generating agent for use in formation of a resist pattern may be employed. The onium cation included in the acid-generating agent [C] is preferably a radiation-sensitive onium cation, more specifically, a sulfonium cation or an iodonium cation. Among them, a triarylsulfonium cation and a diaryliodonium cation are particularly preferred. Specific examples thereof include the same cations exemplified as the cation represented by the aforementioned formula (4) or (5).

No particular limitation is imposed on the organic anion included in the acid-generating agent [C], so long as anion is a compound which can generate an acid upon exposure of the present composition to light. Among such anions, sulfonate anion, imide anion, or methide anion is preferred, with a sulfonate anion including an iodine atom being more preferred.

Specific examples of the organic anion forming the acid-generating agent [C] include anions represented by the following formulas. However, the structure of the organic anion forming the acid-generating agent [C] is not limited to the following structures.

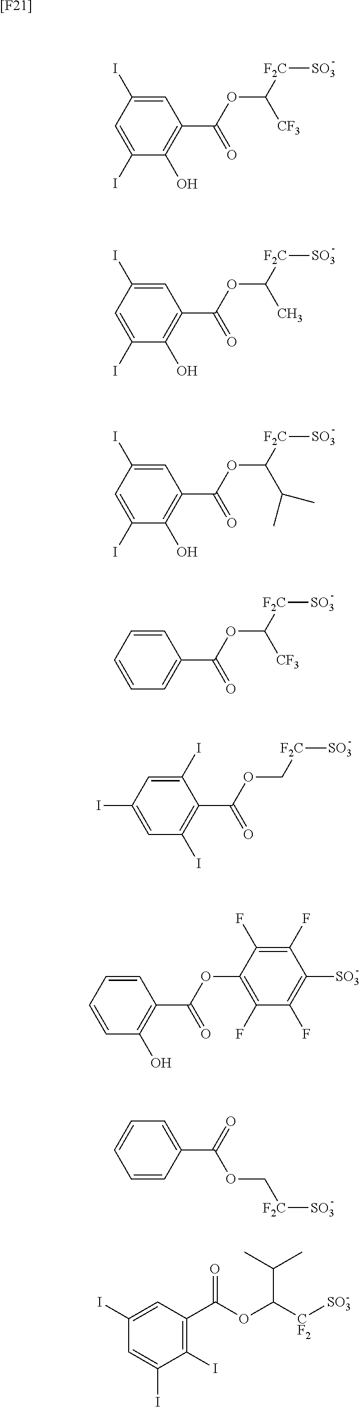

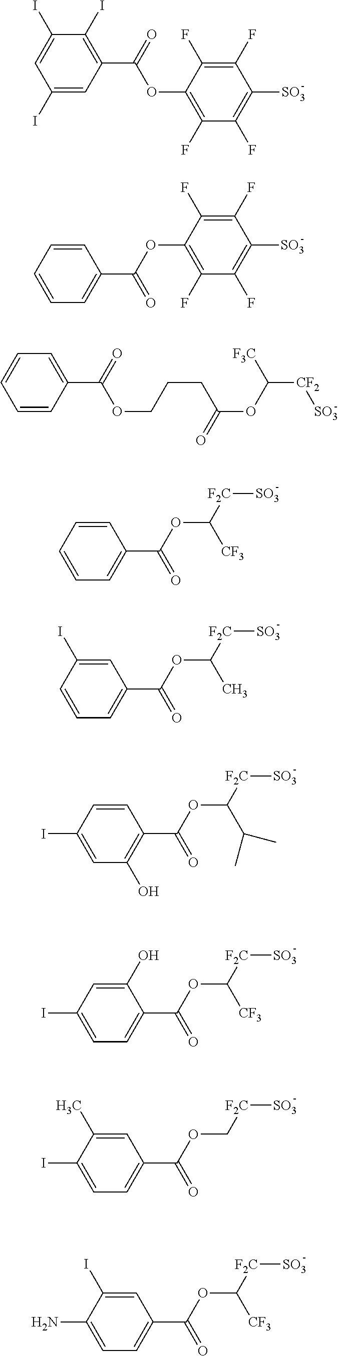

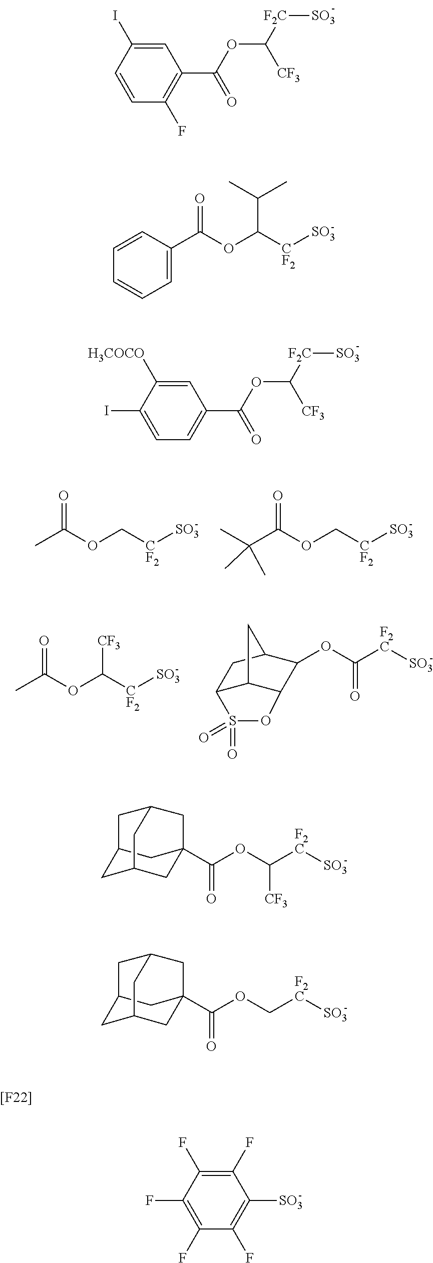

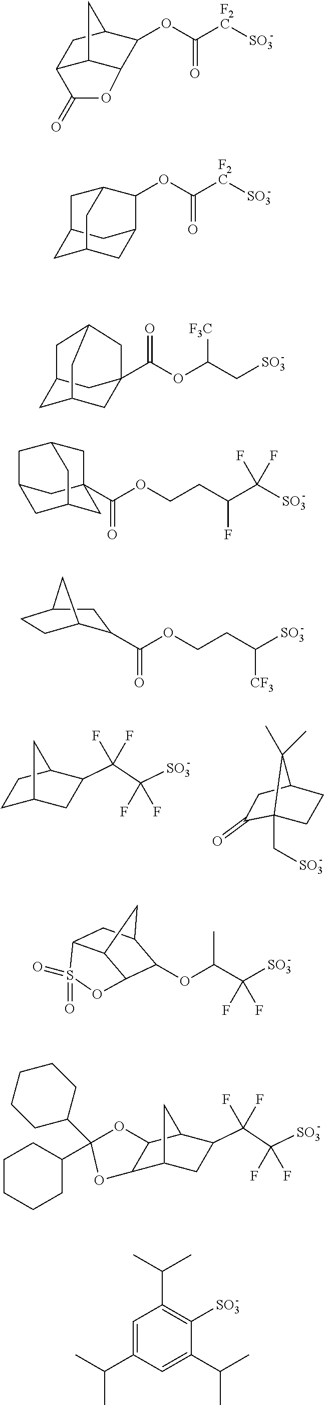

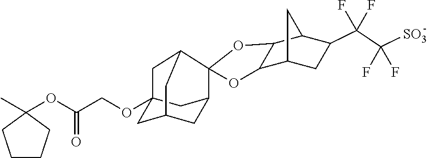

When the polymer [A] includes the structural unit (V), the polymer [A] exerts a function of generating acid in the present composition through light exposure. Thus, even when the present composition does not contain the acid-generating agent [C], a favorable resist pattern can be formed.

When the acid-generating agent [C] is incorporated into the present composition, the relative amount of the acid-generating agent [C], with respect to 100 parts by mass of the polymer [A], is preferably 1 part by mass or more, more preferably 5 parts by mass or more, still more preferably 10 parts by mass or more. Also, the relative amount of the acid-generating agent [C], with respect to 100 parts by mass of the polymer [A], is preferably 50 parts by mass or less, more preferably 40 parts by mass or less, still more preferably 30 parts by mass or less. Controlling the acid-generating agent [C] content to satisfy the above conditions is preferred, since the failure-suppressing effect, LWR performance, and sensitivity of the present composition can be further enhanced. The acid-generating agent [C] may be used singly or in combination of two or more species.

In the present composition, the relative amount of compound (b), with respect to 100 parts by mole of the acid-generating agent [C], is preferably 1 part by mole or more, more preferably 3 part by mole or more, still more preferably 5 parts by mole or more. Also, the relative amount of compound (b), with respect to 100 parts by mole of the acid-generating agent [C], is preferably 200 parts by mole or less, more preferably 100 parts by mole or less, still more preferably 50 parts by mole or less. Controlling the compound (b) content to satisfy the above conditions is preferred, since the effects of improving the sensitivity of the present composition and LWR performance and reducing development failure can be achieved in a well-balanced manner.

<Compound (d)>

An example of the compound (d) is a light-degradable base formed of a radiation-sensitive cation having a structure differing from that of the cation in the formula (1), and an organic anion. As the light-degradable base, there may be used a compound which can generate an acid weaker than the acid generated in the present composition by the acid-generating agent [C] upon exposure of the present composition to light. Specific examples include a compound which can provide a weak acid (preferably carboxylic acid), a sulfonic acid, or sulfonimide, upon exposure to light. In the present composition, the relative amount of the compound (d), with respect to the total amount of the compounds (b) and (d) contained in the present composition, is preferably 3 mol % or less, more preferably 1 mol % or less, still more preferably 0.5 mol % or less.

### <Solvent [E]>

No particular limitation is imposed on the solvent [E], so long as the solvent can dissolve or disperse the components incorporated into the present composition therein. Examples of the solvent [E] include an alcohol, an ether, a ketone, an amide, an ester, and a hydrocarbon.

Examples of the alcohol include C1 to C18 aliphatic monoalcohols such as 4-methyl-2-pentanol and n-hexanol; C3 to C18 alicyclic monoalcohols such as cyclohexanol; C2 to C18 polyhydric alcohols such as 1,2-propylene glycol; and C3 to C19 polyhydric alcohol partial ethers such as propylene glycol monomethyl ether. Examples of the ether include dialkyl ethers such as diethyl ether, dipropyl ether, dibutyl ether, dipentyl ether, diisoamyl ether, dihexyl ether, and diheptyl ether; cyclic ethers such as tetrahydrofuran and tetrahydropyran; and aromatic ring-containing ethers such as diphenyl ether and anisole.

Examples of the ketone include chain ketones such as acetone, methyl ethyl ketone, methyl n-propyl ketone, methyl n-butyl ketone, diethyl ketone, methyl isobutyl ketone, 2-heptanone, ethyl n-butyl ketone, methyl n-hexyl ketone, diisobutyl ketone, and trimethylnonanone; cyclic ketones such as cyclopentanone, cyclohexanone, cycloheptanone, cyclooctanone, and methylcyclohexanone; and 2,4-pentanedione, acetonylacetone, acetophenone, and diacetone alcohol. Examples of the amide include cyclic amides such as N,N′-dimethylimidazolidinone and N-methylpyrrolidone; and chain amides such as N-methylformamide, N,N-dimethylformamide, N,N-diethylformamide, acetamide, N-methylacetamide, N,N-dimethylacetamide, and N-methylpropionamide.

Examples of the ester include monocarboxylic acid ester-type solvents such as n-butyl acetate and ethyl lactate; polyhydric alcohol carboxylates such as propylene glycol acetate; polyhydric alcohol partial ester carboxylates such as propylene glycol monomethyl ether acetate; polybasic carboxylic acid diesters such as diethyl oxalate; carbonates such as dimethyl carbonate and diethyl carbonate; and cyclic esters such as γ-butyrolactone. Examples of the hydrocarbon include C5 to C12 aliphatic hydrocarbons such as n-pentane and n-hexane; and C6 to C16 aromatic hydrocarbons such as toluene and xylene.

Among the above solvents, the solvent [E] preferably includes at least one member selected from the group consisting of the ester and the ketone, more preferably at least one member selected from the group consisting of a polyhydric alcohol partial ether carboxylate and an cyclic ketone. The solvent [E] may be used singly or in combination of two or more species.

### <High-Fluorine Content Polymer [F]>

The high-fluorine content polymer [F](hereinafter may also be referred to simply as a “polymer [F]”) is a polymer having a fluorine atom content (by mass) greater than that of polymer [A]. The polymer [F] is incorporated into the present composition as, for example, a water-repellent additive.

No particular limitation is imposed on the fluorine atom content of the polymer [F], so long as it is greater than the fluorine atom content of the polymer [A]. The fluorine atom content of the polymer [F] is preferably 1 mass % or more, more preferably 4 mass % or more, particularly preferably 7 mass % or more. Also, the fluorine atom content of the polymer [F] is preferably 60 mass % or less, more preferably 40 mass % or less. The fluorine atom content (mass %) of a polymer can be obtained by determining the structure of the polymer through ¹³C-NMR spectrometry or the like and calculating the content based on the structure determined.

When the present composition contains the polymer [F], the relative amount of the polymer [F] in the present composition with respect to 100 parts by mass of polymer [A] is preferably 0.05 parts by mass or more, more preferably 0.1 parts by mass or more, still more preferably 0.5 parts by mass or more. Also, the polymer [F] content with respect to 100 parts by mass of polymer [A] is preferably 10 parts by mass or less, more preferably 5 parts by mass or less, still more preferably 3 parts by mass or less. Notably, the present composition may contain the polymer [F]singly or in combination of two or more species.

### <Additional and Optional Component>

The present composition may further contain a component which differs from the aforementioned polymer [A], acid-generator [B], solvent [E], and high-fluorine content polymer [F](hereinafter the component may also be referred to as “additional and optional component”). Examples of the additional and optional component include a surfactant, a compound having an alicyclic skeleton (e.g., 1-adamantanecarboxylic acid, 2-adamantanone, or t-butyl deoxycholate), a sensitizer, a localization accelerator, and a nitrogen-containing compound. Notably, in the present specification, the compound (b), the compound (d), and the nitrogen-containing compound may also be referred collectively to an “acid diffusion control agent [D].” So long as the effect of the present disclosure are not impaired, the additional and optional component content of the present composition may be appropriately set depending on the property of the component.

There has not been completely elucidated a reason why the present composition containing the polymer [A] and the compound (b) has excellent sensitivity and LWR performance and can form a resist pattern in which generation of development failure is suppressed. However, the following possible reasons can be conceived. In one conceivable reason, an iodine atom incorporated into the compound (b) exhibits particularly high light (particularly EUV light) absorption efficiency, and decomposition of the acid-generator [B](i.e., acid-generating agent and light-degradable base) occurring in the present composition upon exposure to light is promoted, leading to well-balanced improvement in the sensitivity and LWR performance of the radiation-sensitive composition. In another conceivable reason, the compound (b) is considered to have higher basicity and hydrophobicity as compared with a component, for example, an N-carbonylsulfonamide-type light-degradable base, whereby the compound (b) easily forms a pseudo-3-dimensional network with a polar group (e.g., a phenolic hydroxy group or the like in the structural unit (II)) present in the polymer [A]. In addition, by virtue of high hydrophobicity, the solubility of the composition in a developer decreases, to thereby effectively suppress dissolution of a light-unexposed part in the developer.

### <Method of Producing Radiation-Sensitive Composition>

The present composition may be produced through, for example, the following procedure: mixing the polymer [A] and the acid-generator [B] with an optional component such as the solvent [E] at a desired ratio and filtering the resultant mixture preferably by means of a filter (e.g., a filter having a pore size of about 0.2 m) or the like. The solid content of the present composition is preferably 0.1 mass % or more, more preferably 0.5 mass % or more, still more preferably 1 mass % or more. Also, the solid content of the present composition is preferably 50 mass % or less, more preferably 20 mass % or less, still more preferably 5 mass % or less. Adjusting the solid content of the present composition to satisfy the above conditions is preferred, since coatability of the composition can be enhanced, to thereby obtain a resist pattern having a suitable shape.

The thus-obtained present composition may also be used as a composition for forming a positive pattern, which is employed for pattern formation by use of an alkaline developer. Alternatively, the present composition may be used as a composition for forming a negative pattern by use of a developer containing organic solvent.

### <Method of Forming Resist Pattern>

The resist pattern formation method of the present disclosure includes a step of applying the present composition on one surface of a substrate (hereinafter may also be referred to as a “application step”), a step of exposing to light a resist film obtained in the application step (hereinafter may also be referred to as a “light-exposure step”), and a step of developing the light-exposed resist film (hereinafter may also be referred to as a “development step”). Examples of the pattern obtained through the resist pattern formation method of the present disclosure include a line-and-space pattern and a hole pattern. Since a resist film is formed by use of the present composition in the resist pattern formation method of the present disclosure, a resist pattern which exhibits excellent sensitivity and lithographic characteristics and which has few development faults can be formed. The steps will next be described in detail.

### [Application Step]

In this step, the present composition is applied onto one surface of a substrate, to thereby form a resist film on the substrate. A conventionally known substrate can be used as a substrate on which resist film is to be formed. Examples of the substrate include a silicon wafer and a wafer coated with silicon dioxide or aluminum. For example, an organic or inorganic anti-reflection film disclosed in, for example, Japanese Patent Publication (kokoku) No. 1994-12452 or Japanese Patent Application laid-Open (kokai) No. 1984-93448 may be formed on a substrate to be used. Examples of the method of applying the present composition include spin coating, flow casting, and roller coating. After application, the applied composition may be subjected to soft baking (SB) so as to evaporate the solvent remaining in the coating film. The temperature of SB is preferably 60° C. or higher, more preferably 80° C. or higher. Also, the temperature of SB is preferably 140° C. or lower, more preferably 120° C. or lower. The time of SB is preferably 5 seconds or longer, more preferably 10 seconds or longer, and preferably 600 seconds or shorter, more preferably 300 seconds or shorter. The average thickness of the formed resist film is preferably 10 to 1,000 nm, more preferably 20 to 500 nm.

### [Light-Exposure Step]

In this step, the resist film formed through the above application step is exposed to light. In the light exposure, the resist film is irradiated with radiation by the mediation of a photomask or, in some cases, a liquid immersion medium such as water. The radiation is selected in accordance with the line width of a target pattern, and examples thereof include electromagnetic waves such as visible light, a UV ray, a far-UV ray, an extreme UV (EUV) ray, an X-ray, and a γ-ray; and charged particle rays such as an electron beam and an C-ray. Among them, the radiation applied to the resist film formed from the present composition is preferably a far-UV ray, an EUV ray, or an electron beam, more preferably ArF excimer laser light (wavelength: 193 nm), KrF excimer laser light (wavelength: 248 nm), an EUV ray, or an electron beam, still more preferably ArF excimer laser light, an EUV ray, or an electron beam, yet more preferably an EUV ray or an electron beam, particularly preferably an EUV ray.

After completion of the above light exposure, post exposure baking (PEB) is preferably performed so as to accelerate dissociation of an acid-releasable group of the polymer [A] and other components in the light-exposed part of the resist film. Through PEB, the difference in dissolution performance with respect to a developer between the exposed part and the unexposed part can be increased. The temperature at PEB is preferably 50° C. or higher, more preferably 80° C. or higher, and preferably 180° C. or lower, more preferably 130° C. or lower. The time of PEB is preferably 5 seconds or longer, more preferably 10 seconds or longer, and preferably 600 seconds or shorter, more preferably 300 seconds or shorter.

### [Development Step]

In this step, the resist film which has been exposed to light in the above step is developed, whereby a resist pattern of interest can be formed. Generally, after development, the developed film is washed with a rinse liquid (e.g., water or alcohol) and then dried. The development method employed in the development step may be development with alkali or with organic solvent.

Examples of the developer employed in the alkali development include aqueous alkaline solutions in which at least one species from among alkaline compounds such as sodium hydroxide, potassium hydroxide, sodium carbonate, sodium silicate, sodium metasilicate, aqueous ammonia, ethylamine, n-propylamine, diethylamine, di-n-propylamine, triethylamine, methyldiethylamine, ethyldimethylamine, triethanolamine, tetramethylammonium hydroxide (TMAH), pyrrole, piperidine, choline, 1,8-diazabicyclo[5.4.0]-7-undecene, 1,5-diazabicyclo[4.3.0]-5-nonene, and the like is dissolved. Among such alkaline solutions, an aqueous TMAH solution is preferred, with 2.38-mass % aqueous TMAH solution being more preferred.

In the case of development with an organic solvent, examples of the developer include of organic solvents such as hydrocarbnons, ethers, esters, ketones, and alcohols; and one or more of solvents each containing any of the above organic solvents. Examples of the organic solvent employed as the developer include the solvents as exemplified in relation to the solvent [E] of the present composition. Among them, esters and ketones are preferred. Among the esters, acetate esters are preferred, with n-butyl acetate being more preferred. Among the ketones, chain ketones are preferred, with 2-heptanone being more preferred. The organic solvent content of the developer is preferably 80 mass % or more, more preferably 90 mass % or more, still more preferably 95 mass % or more, particularly preferably 99 mass % or more. Examples of developer components other than organic solvent include water and silicone oil.

Examples of the development method include a dipping method (i.e., dipping a substrate in a bath filled with a developer for a specific time); a paddle method (i.e., putting a developer in a substrate to form a drop by surface tension and allowing the substrate to stand for a specific time); a spray method (i.e., spraying a developer onto a substrate); and a dynamic dispense method (i.e., continuously jetting a developer at a specific speed to a substrate rotating at a specific speed through a developer jetting nozzle with scanning).

### EXAMPLES

The present disclosure will next be described in detail by way of examples, which should not be construed as limiting the disclosure thereto. Unless otherwise specified, the units “part(s)” and “%” in the Examples are on a mass basis. Measurements in the Examples and Comparative Examples were conducted by the following procedures.

### [Weight Average Molecular Weight (Mw) and Number Average Molecular Weight (Mn)]

The weight average molecular weight (Mw) and the number average molecular weight (Mn) of a polymer were determined through gel permeation chromatography (GPC) with GPC columns (G2000HXL×2, G3000HXL×1, and G4000HXL×1) (products of Tosoh Corp.) under the following conditions.
Eluent: tetrahydrofuran (product of FUJIFILM Wako Pure Chemical Corporation)
Flow rate: 1.0 mL/min
Sample concentration: 1.0 mass %
Sample injection: 100 L
Column temperature: 40° C.
Detector: differential refractometer
Standard: monodispersed polystyrene

### <Synthesis of Polymer [A]>

The following monomers were used in synthesis of the polymers.

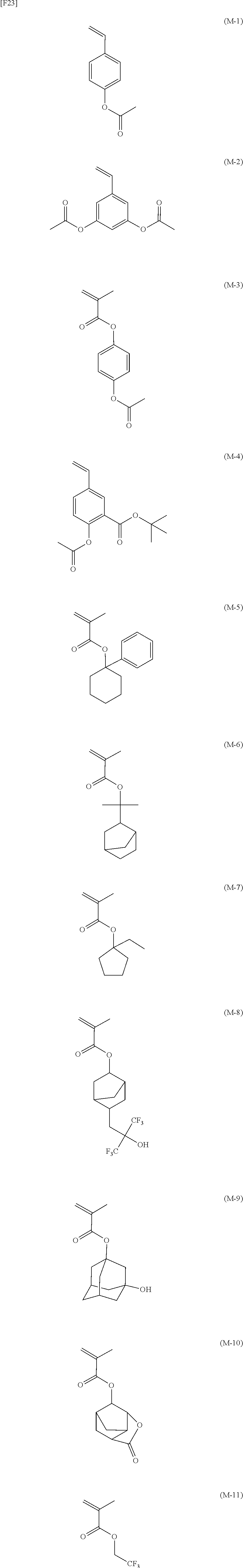

In the following Synthesis Examples, unless otherwise specified, the unit “part(s) by mass” is based on the total mass of the monomers used as 100 parts by mass. The unit “mol %” shown in parenthesis is based on the total amount by mole of the monomers used as 100 mol %.

### Synthesis Example 1: Synthesis of Polymer (A-1)

Compound (M-1) (3.33 g, 45 mol %), compound (M-5) (5.02 g, 45 mol %), and compound (M-8) (1.65 g, 10 mol %) were dissolved in propylene glycol monomethyl ether (200 parts by mass). To the solution, 2,2′-azobis(methyl isobutyrate) (0.736 g, 7 mol %) serving as an initiator was added, to thereby prepare a monomer solution. Separately, propylene glycol monomethyl ether (100 parts by mass) was added to a vacant reaction container, and the contents were heated at 85° C. under stirring. Subsequently, the above-prepared monomer solution was added dropwise to the reaction container over 3 hours. After addition, the mixture was further heated at 85° C. for 3 hours. After completion of the polymerization reaction, the reaction mixture was cooled to room temperature. The polymer solution was added dropwise to n-hexane (1,000 parts by mass), whereby the polymer was refined through solidification. Propylene glycol monomethyl ether (150 parts by mass) was added again to the thus-obtained polymer. Next, methanol (150 parts by mass), triethylamine (1.5 mole equivalents to the amount of the compound (M-1)), and water (1.5 mole equivalents to the amount of the compound (M-1)) were added thereto. While the resultant mixture was refluxed at a boiling temperature, hydrolysis was performed for 8 hours. After completion of hydrolysis reaction, the solvent and triethylamine were distilled off under reduced pressure. The thus-obtained polymer was dissolved in acetone (150 parts by mass), and the solution was added dropwise to water (2,000 parts by mass), to thereby solidify the polymer. The thus-formed white powder was separated through filtration and dried at 50° C. for 17 hours, to thereby yield a polymer (A-1) in the form of white powder at a considerable yield.

### Synthesis Examples 2 to 8: Synthesis of Polymers (A-2) to (A-8)

The procedure of Synthesis Example 1 was repeated, except that monomers shown in Table 1 were employed, to thereby yield polymers (A-2) to (A-8), respectively. The amounts of triethylamine and water were adjusted to 1.5 mole equivalents to the amounts of the compound (M-1), the compound (M-3), and the compound (M-4), respectively, and to 3.0 mole equivalents to the amount of the compound (M-2). In the case where two members of the compounds (M-1) to (M-4) were used, the total amount thereof is shown in the table.

### Synthesis Example 9: Synthesis of Polymer (A-9)

Compound (M-7) (9.01 g, 50 mol %) and compound (M-10) (10.99 g, 50 mol %) were dissolved in 2-butanone (200 parts by mass), and azobisisobutyronitrile (0.654 g, 4 mol %) was further dissolved therein, to thereby prepare a monomer solution. Separately, 2-butanone (100 parts by mass) was added to a 200-mL three-neck flask. In an nitrogen atmosphere, the contents of the flask were heated at 80° C. under stirring. The above-prepared monomer solution was added dropwise to the flask over 3 hours. After completion of addition, the contents were further heated at 80° C. for 3 hours. After completion of polymerization reaction, the reaction mixture was cooled to room temperature and added to methanol (300 g). The deposited solid was separated through filtration, and the separated solid was washed twice with methanol (60 mL). The washed solid was separated through filtration and dried at 50° C. for 15 hours under reduced pressure, to thereby yield a polymer (A-9).

Table 1 shows the types and amounts of monomers employed in the Synthesis Examples, and the Mw and Mw/Mn of each of the produced polymers.

TABLE 1

| | | Monomer providing | | Monomer providing | | Monomer providing | | Monomer providing | | | |
|---|---|---|---|---|---|---|---|---|---|---|---|
| | | structural unit (I) | | structural unit (II) | | structural unit (III) | | structural unit (IV) | | | |
| | Polymer | | Amount | | Amount | | Amount | | Amount | | |
| | [A] | Type | (mol %) | Type | (mol %) | Type | (mol %) | Type | (mol %) | Mw | Mw/Mn |
| Synthesis | A-1 | M-5 | 45 | M-1 | 45 | — | — | M-8 | 10 | 7500 | 1.5 |
| Example 1 | | | | | | | | | | | |
| Synthesis | A-2 | M-5 | 55 | M-1 | 25 | — | — | — | — | 5300 | 1.4 |
| Example 2 | | | | M-2 | 20 | | | | | | |
| Synthesis | A-3 | M-6 | 55 | M-2 | 45 | — | — | — | — | 4200 | 1.3 |
| Example 3 | | | | | | | | | | | |
| Synthesis | A-4 | M-7 | 40 | M-2 | 30 | — | — | — | — | 6800 | 1.6 |
| Example 4 | | | | M-3 | 30 | | | | | | |
| Synthesis | A-5 | M-7 | 70 | M-1 | 30 | — | — | — | — | 5700 | 1.5 |
| Example 5 | | | | | | | | | | | |
| Synthesis | A-6 | M-6 | 50 | M-1 | 35 | — | — | M-9 | 15 | 8500 | 1.5 |
| Example 6 | | | | | | | | | | | |
| Synthesis | A-7 | M-4 | 60 | M-1 | 30 | M-10 | 10 | — | — | 6300 | 1.5 |
| Example 7 | | | | | | | | | | | |
| Synthesis | A-8 | M-7 | 35 | M-1 | 35 | — | — | — | — | 4800 | 1.4 |
| Example 8 | | M-4 | 30 | | | | | | | | |
| Synthesis | A-9 | M-7 | 50 | — | — | M-10 | 50 | — | — | 10600 | 1.6 |
| Example 9 | | | | | | | | | | | |

### Synthesis of Polymer [F]

### [Synthesis Example 10](Synthesis of Polymer (F-1))

Compound (M-7) (7.17 g, 70 mol %) and compound (M-11) (2.83 g, 30 mol %) were dissolved in 2-butanone (100 parts by mass). To the solution, azobisisobutyronitrile (0.461 g, 5 mol %) serving as an initiator was further added, to thereby prepare a monomer solution. Separately, 2-butanone (50 parts by mass) was added to a vacant reaction container. The atmosphere of the container was purged with nitrogen for 30 minutes. Subsequently, the inside temperature of the reaction container was adjusted to 80° C., and the above-prepared monomer solution was added dropwise to the container over 3 hours under stirring. Polymerization reaction was conducted for 6 hours, wherein the time of start of dropwise addition was employed as the time of initiating polymerization reaction. After completion of polymerization reaction, the reaction mixture was water-cooled to 30° C. or lower. The cooled reaction mixture was transferred to a separatory funnel, and uniformly diluted with hexane (150 parts by mass). Then, methanol (600 parts by mass) and water (30 parts by mass) were added to the funnel, and the contents were mixed, followed by allowing the mixture to stand for 30 minutes. The lower layer was recovered, and the solvent was changed to propylene glycol monomethyl ether acetate, to thereby yield a solution containing a polymer (F-1) in propylene glycol monomethyl ether acetate.

### Synthesis of Acid Diffusion Control Agent [D]

### Synthesis Example 11: Synthesis of Compound (D-1)

A compound (D-1) was synthesized through the following reaction scheme.

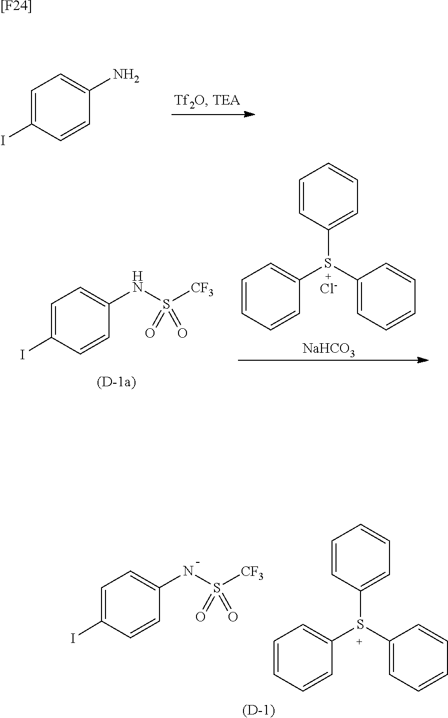

To a reaction container, 4-iodoaniline (5.0 mmol), triethylamine (7.5 mmol), and chloromethylene (50 mL) were added, and the contents were mixed and cooled to −30° C. Subsequently, trifluoromethanesulfonic anhydride (6.0 mmol) was added dropwise thereto. The contents were stirred at −30° C. for 1 hour, and reaction was terminated by adding 1N HCl (50 mL). The mixture was subjected to liquid separation, and the organic layer was dried over sodium sulfate anhydrate, followed by filtration. The product was purified through silica gel column chromatography, to thereby yield an intermediate (D-1a) at a yield of 55%.

The intermediate (D-1a) (2.3 mmol), sodium hydrogen carbonate (3.3 mmol), triphenylsulfonium chloride (2.3 mmol), chloromethylene (20 mL), and distilled water (20 mmol) were mixed together under stirring at room temperature for 3 hours. After completion of reaction, the mixture was subjected to liquid separation, and the organic layer was dried over sodium sulfate anhydrate, followed by filtration. The solvent was distilled out, to thereby yield the compound (D-1) at a yield of 92%.

### Synthesis Examples 12 to 17: Synthesis of Compounds (D-2) to (D-7)

By choosing an appropriate precursor and the same formulation as employed in Synthesis Example 11, compounds represented by the following formulas (D-2) to (D-7) (compounds (D-2) to (D-7)) were synthesized.

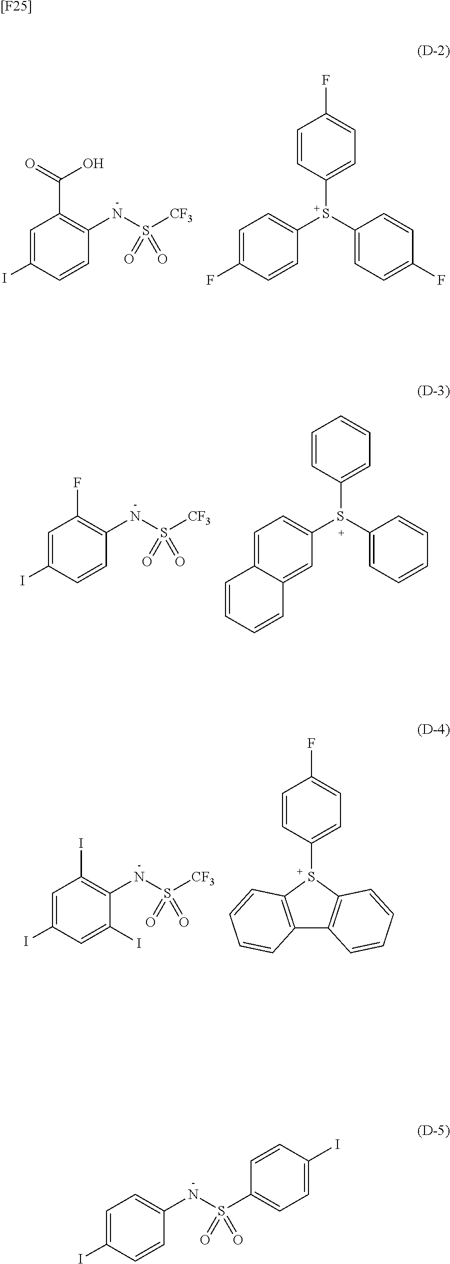

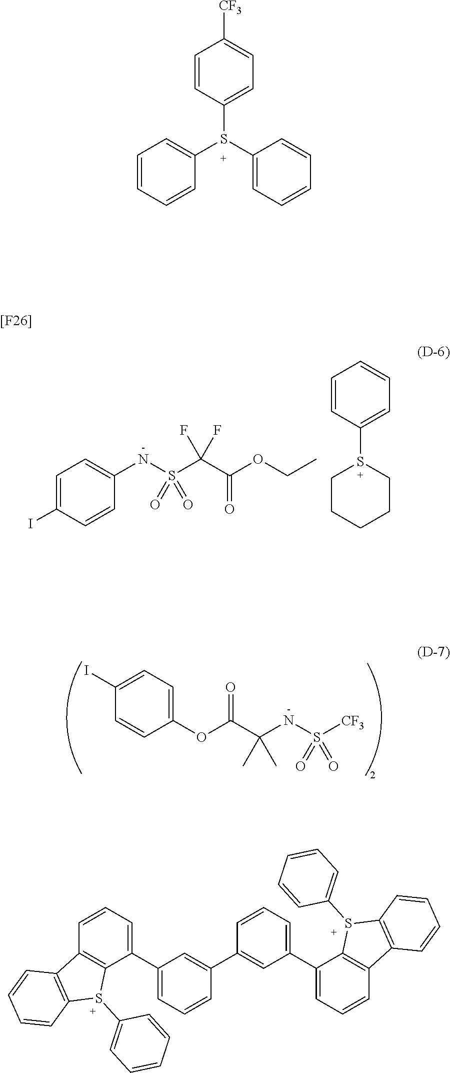

### Synthesis Example 18: Synthesis of Compound (D-8)

A compound (D-8) was synthesized through the following reaction scheme.

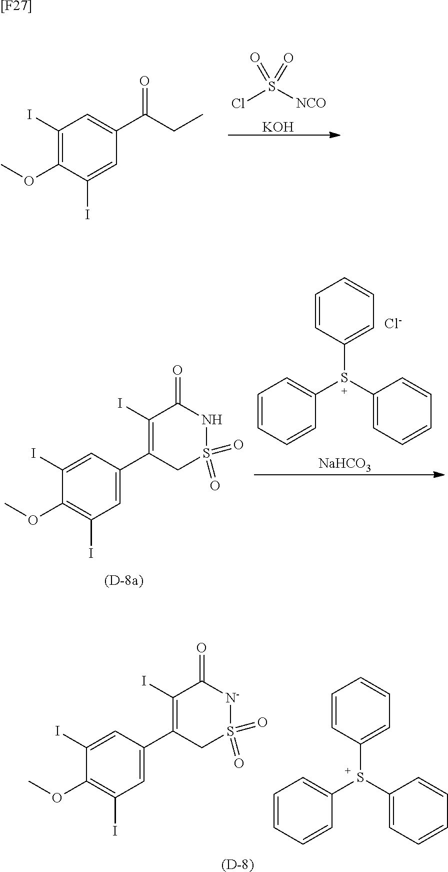

Chlorosulfonyl isocyanate (22 mmol) was added dropwise to a mixture of diethyl ether (10 mL) and 3,5-diiodo-4-methoxypropiophenone (6.0 mmol), and the resultant mixture was stirred at room temperature for 2 hours. 4N Aqueous potassium hydroxide was added thereto, to thereby adjust the pH to 11 or higher, and formed precipitates were recovered through filtration. The solid was washed with a small amount of water and ethyl acetate and dried under reduced pressure, to thereby yield an intermediate (D-8a) at a yield of 40%.

The intermediate (D-8a) (2.4 mmol), sodium hydrogen carbonate (3.6 mmol), triphenylsulfonium chloride (2.4 mmol), chloromethylene (20 mL), and distilled water (20 mmol) were mixed together under stirring at room temperature for 3 hours. After completion of reaction, the mixture was subjected to liquid separation, and the organic layer was dried over sodium sulfate anhydrate, followed by filtration. The solvent was distilled out, to thereby yield the compound (D-8) at a yield of 88%.

### Synthesis Examples 19 and 20: Synthesis of Compounds (D-9) and (D-10)

By choosing an appropriate precursor and the same formulation as employed in Synthesis Example 18, compounds represented by the following formulas (D-9) and (D-10) (compounds (D-9) and (D-10)) were synthesized.

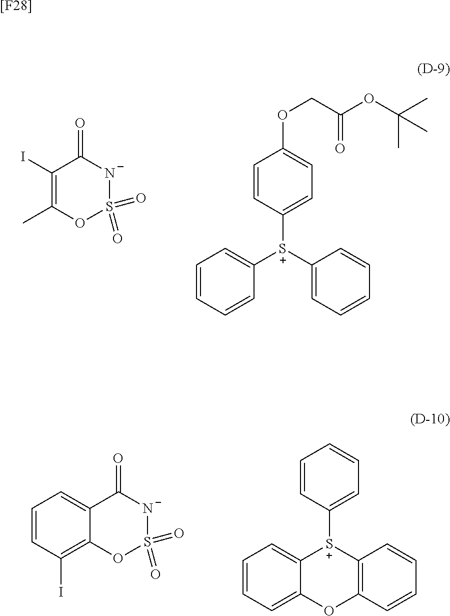

### <Preparation of Radiation-Sensitive Resin Compositions>

The additional acid diffusion control agent [DD], acid-generating agent [C], and solvent [E] used in preparation of the radiation-sensitive resin compositions of the below-mentioned Examples and Comparative Examples are as follows.

Additional acid diffusion control agent [DD] Compounds represented by the following formulas (DD-1) to (DD-4) (compounds (DD-1) to (DD-4)) were used as additional acid diffusion control agents.

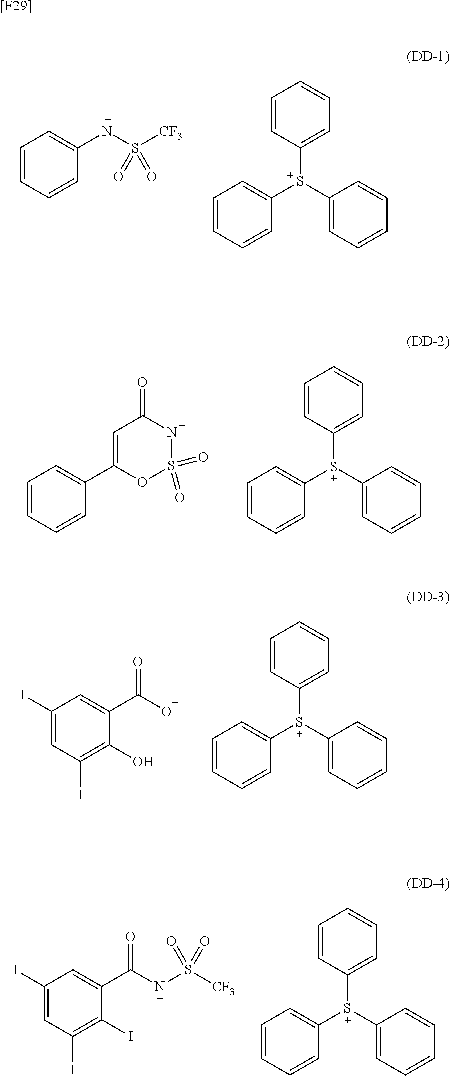

### Acid-Generating Agent [C]

Compounds represented by the following formulas (C-1) to (C-12) (compounds (C-1) to (C-12)) were used as acid-generating agents.

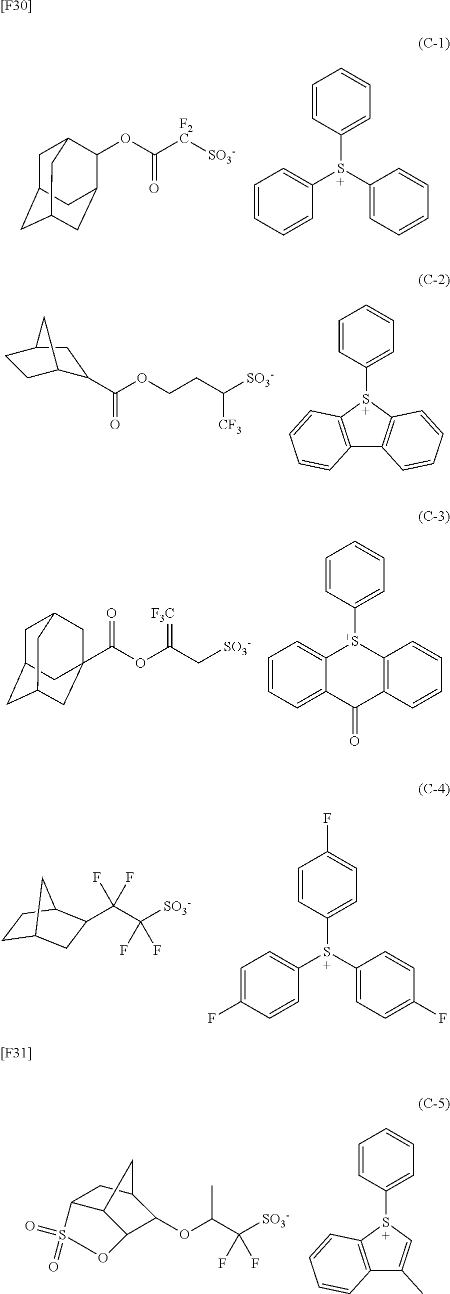

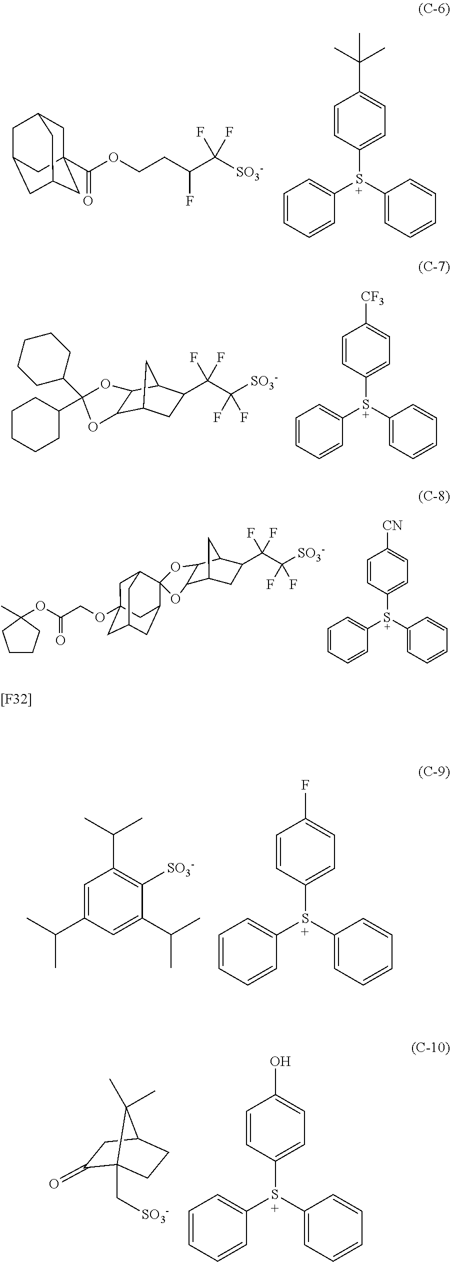

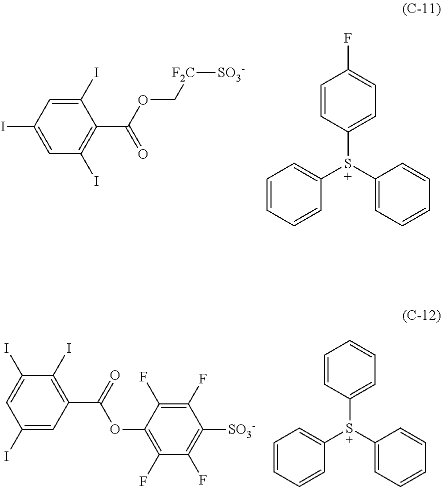

### Solvent [E]

E-1: propylene glycol monomethyl ether acetate
E-2: propylene glycol monomethyl ether

### Example 1

The polymer (A-1) (100 parts by mass), the polymer (F-1) (1 part by mass, as solid), the compound (C-1) (22 parts by mass), the compound (D-1) (20 mol %, with respect to the compound (C-1)), the solvent (E-1) (5,500 parts by mass), and the solvent (E-2) (1,500 parts by mass) were mixed together, to thereby prepare a radiation-sensitive resin composition (R-1). The amount of solvent (E-1) shown in Tables 2 and 3 is a total amount thereof with the amount of propylene glycol monomethyl ether acetate contained in the polymer (F-1) solution obtained in Synthesis Example 10. In Tables 2 and 3, the amount of acid diffusion control agent [D] refers to a ratio (mol %) of the amount thereof to the amount of acid-generating agent [C].

### Examples 2 to 31 and Comparative Examples 1 to 5

The procedure of Example 1 was repeated, except that the types and amounts of the components were changed as shown in Tables 2 and 3, to thereby prepare radiation-sensitive resin compositions (R-2) to (R-31), and (CR-1) to (CR-5).

TABLE 2

| | | | | | | Acid-generating | | Acid diffusion | | | |
|---|---|---|---|---|---|---|---|---|---|---|---|
| | Radiation- | Polymer [A] | | Polymer [F] | | agent [C] | | control agent [D] | | Solvent [E] | |
| | sensitive | | Amount | | Amount | | Amount | | Amount | | Amount |
| | resin | | (part(s) | | (part(s) | | (part(s) | | (part(s) | | (part(s) |
| | composition | Type | by mass) | Type | by mass) | Type | by mass) | Type | by mass) | Type | by mass) |
| Example 1 | R-1 | A-1 | 100 | F-1 | 1 | C-1 | 22 | D-1 | 20 | E-1/E-2 | 5500/1500 |
| Example 2 | R-2 | A-1 | 100 | F-1 | 1 | C-1 | 22 | D-2 | 20 | E-1/E-2 | 5500/1500 |
| Example 3 | R-3 | A-1 | 100 | F-1 | 1 | C-1 | 22 | D-3 | 20 | E-1/E-2 | 5500/1500 |
| Example 4 | R-4 | A-1 | 100 | F-1 | 1 | C-1 | 22 | D-4 | 20 | E-1/E-2 | 5500/1500 |
| Example 5 | R-5 | A-1 | 100 | F-1 | 1 | C-1 | 22 | D-5 | 20 | E-1/E-2 | 5500/1500 |
| Example 6 | R-6 | A-1 | 100 | F-1 | 1 | C-1 | 22 | D-6 | 20 | E-1/E-2 | 5500/1500 |
| Example 7 | R-7 | A-1 | 100 | F-1 | 1 | C-1 | 22 | D-7 | 10 | E-1/E-2 | 5500/1500 |
| Example 8 | R-8 | A-1 | 100 | F-1 | 1 | C-1 | 22 | D-8 | 20 | E-1/E-2 | 5500/1500 |
| Example 9 | R-9 | A-1 | 100 | F-1 | 1 | C-1 | 22 | D-9 | 20 | E-1/E-2 | 5500/1500 |
| Example 10 | R-10 | A-1 | 100 | F-1 | 1 | C-1 | 22 | D-10 | 20 | E-1/E-2 | 5500/1500 |
| Example 11 | R-11 | A-2 | 100 | F-1 | 1 | C-1 | 22 | D-1 | 20 | E-1/E-2 | 5500/1500 |
| Example 12 | R-12 | A-3 | 100 | F-1 | 1 | C-1 | 22 | D-1 | 20 | E-1/E-2 | 5500/1500 |
| Example 13 | R-13 | A-4 | 100 | F-1 | 1 | C-1 | 22 | D-1 | 20 | E-1/E-2 | 5500/1500 |
| Example 14 | R-14 | A-5 | 100 | F-1 | 1 | C-1 | 22 | D-1 | 20 | E-1/E-2 | 5500/1500 |
| Example 15 | R-15 | A-6 | 100 | F-1 | 1 | C-1 | 22 | D-1 | 20 | E-1/E-2 | 5500/1500 |
| Example 16 | R-16 | A-7 | 100 | F-1 | 1 | C-1 | 22 | D-1 | 20 | E-1/E-2 | 5500/1500 |
| Example 17 | R-17 | A-8 | 100 | F-1 | 1 | C-1 | 22 | D-1 | 20 | E-1/E-2 | 5500/1500 |
| Example 18 | R-18 | A-1 | 100 | F-1 | 1 | C-2 | 22 | D-1 | 20 | E-1/E-2 | 5500/1500 |
| Example 19 | R-19 | A-1 | 100 | F-1 | 1 | C-3 | 22 | D-1 | 20 | E-1/E-2 | 5500/1500 |
| Example 20 | R-20 | A-1 | 100 | F-1 | 1 | C-4 | 22 | D-1 | 20 | E-1/E-2 | 5500/1500 |
| Example 21 | R-21 | A-1 | 100 | F-1 | 1 | C-5 | 22 | D-1 | 20 | E-1/E-2 | 5500/1500 |
| Example 22 | R-22 | A-1 | 100 | F-1 | 1 | C-6 | 22 | D-1 | 20 | E-1/E-2 | 5500/1500 |
| Example 23 | R-23 | A-1 | 100 | F-1 | 1 | C-7 | 22 | D-1 | 20 | E-1/E-2 | 5500/1500 |
| Example 24 | R-24 | A-1 | 100 | F-1 | 1 | C-8 | 22 | D-1 | 20 | E-1/E-2 | 5500/1500 |
| Example 25 | R-25 | A-1 | 100 | F-1 | 1 | C-9 | 22 | D-1 | 20 | E-1/E-2 | 5500/1500 |
| Example 26 | R-26 | A-1 | 100 | F-1 | 1 | C-10 | 22 | D-1 | 20 | E-1/E-2 | 5500/1500 |
| Example 27 | R-27 | A-1 | 100 | F-1 | 1 | C-11 | 22 | D-1 | 20 | E-1/E-2 | 5500/1500 |
| Example 28 | R-28 | A-1 | 100 | F-1 | 1 | C-12 | 22 | D-1 | 20 | E-1/E-2 | 5500/1500 |
| Example 29 | R-29 | A-1 | 100 | F-1 | 1 | C-1/C-11 | 11/11 | D-1 | 20 | E-1/E-2 | 5500/1500 |
| Comparative | CR-1 | A-1 | 100 | F-1 | 1 | C-1 | 22 | DD-1 | 20 | E-1/E-2 | 5500/1500 |
| Example 1 | | | | | | | | | | | |
| Comparative | CR-2 | A-1 | 100 | F-1 | 1 | C-1 | 22 | DD-2 | 20 | E-1/E-2 | 5500/1500 |
| Example 2 | | | | | | | | | | | |
| Comparative | CR-3 | A-1 | 100 | F-1 | 1 | C-1 | 22 | DD-3 | 20 | E-1/E-2 | 5500/1500 |
| Example 3 | | | | | | | | | | | |
| Comparative | CR-4 | A-1 | 100 | F-1 | 1 | C-1 | 22 | DD-4 | 20 | E-1/E-2 | 5500/1500 |
| Example 4 | | | | | | | | | | | |

TABLE 3

| | | | | | | Acid-generating | | Acid diffusion | | | |
|---|---|---|---|---|---|---|---|---|---|---|---|
| | Radiation- | Polymer [A] | | Polymer [F] | | agent [C] | | control agent [D] | | Solvent [E] | |
| | sensitive | | Amount | | Amount | | Amount | | Amount | | Amount |
| | resin | | (part(s) | | (part(s) | | (part(s) | | (part(s) | | (part(s) |
| | composition | Type | by mass) | Type | by mass) | Type | by mass) | Type | by mass) | Type | by mass) |
| Example 30 | R-30 | A-9 | 100 | F-1 | 1 | C-7 | 18 | D-1 | 20 | E-1/E-2 | 2400/800 |
| Example 31 | R-31 | A-9 | 100 | F-1 | 1 | C-7 | 18 | D-9 | 20 | E-1/E-2 | 2400/800 |
| Comparative | CR-5 | A-9 | 100 | F-1 | 1 | C-7 | 18 | DD-1 | 20 | E-1/E-2 | 2400/800 |
| Example 5 | | | | | | | | | | | |

### <Formation of Resist Pattern (1)>

A 12-inch silicon wafer having an under-layer film having a thickness of 20 nm (AL-412, product of Brewer Science, Inc.) was used. Onto the surface of the wafer, each of the above-prepared radiation-sensitive resin compositions (R-1) to (R-29) and (CR-1) to (CR-4) was applied by means of a spin coater (CLEAN TRACK ACT12, product of Tokyo Electron Ltd.). The wafer was subjected to soft baking (SB) at 100° C. for 60 seconds and then cooled at 23° C. for 30 seconds, to thereby form a resist film having a thickness of 30 nm. Subsequently, the resist film was irradiated with EUV light by means of an EUV exposure device (NXE3300, product of ASML, NA=0.33, lighting condition: Conventional s=0.89). After the EUV light exposure, the resist film was subjected to PEB (post exposure baking) at 100° C. for 60 seconds. Then, development was performed by use of 2.38 mass % aqueous TMAH at 23° C. for 30 seconds, to thereby form a positive-type line-and-space pattern (pitch: 50 nm, line: 25 nm).

### <Evaluation of EUV Resist>

Physical properties of each of the resist patterns formed in the aforementioned <Formation of resist pattern (1)> was measured through the following procedures, to thereby evaluate the sensitivity of each radiation-sensitive resin composition, LWR performance, and failure density. Table 4 shows the results.

### [Sensitivity]

In the aforementioned <Formation of resist pattern (1)>, a dose which can form a line pattern having a width of 25 nm was employed as an optimum dose, serving as a sensitivity index (mJ/cm²). A sensitivity index lower than 60 mJ/cm² was evaluated as “good”, and a sensitivity index of 60 mJ/cm² or higher was evaluated as “bad”.

### [LWR Performance]

Each resist pattern was observed from above under a scanning electron microscope (CG-5000, product of Hitachi High Technology Co., Ltd.), and line width was measured at 800 points selected at random. Variation in width (3(7) was determined to serve as LWR performance (nm). The smaller the variation in width (3(7), the smaller the variation in line width in a long period (i.e., the more excellent the LWR performance). LWR performance was evaluated as “good” when the variation in width (3(7) was smaller than 4.0 nm, and as “bad” when the variation in width (3(7) was 4.0 nm or greater.

### [Failure Density]

The above-formed line-and-space pattern (pitch: 50 nm, line: 25 nm) was subjected to a failure inspection by means of KLA2925 (product of KLA). The smaller the measurement, the more excellent the failure density. The failure density was evaluated as “good” when the measurement was less than 50 ea/cm², and as “bad” when the measurement was 50 ea/cm² or more.

TABLE 4

| | Radiation- | | | Failure |
|---|---|---|---|---|
| | sensitive resin | Sensitivity | LWR | density |
| | composition | (mJ/cm²) | (nm) | (ea/cm²) |
| Example 1 | R-1 | 50 | 3.2 | 32 |
| Example 2 | R-2 | 45 | 3.4 | 28 |
| Example 3 | R-3 | 51 | 3.3 | 30 |
| Example 4 | R-4 | 55 | 2.9 | 40 |
| Example 5 | R-5 | 52 | 2.8 | 26 |
| Example 6 | R-6 | 52 | 3.0 | 46 |
| Example 7 | R-7 | 54 | 2.7 | 38 |
| Example 8 | R-8 | 48 | 3.2 | 27 |
| Example 9 | R-9 | 47 | 3.1 | 41 |
| Example 10 | R-10 | 51 | 3.0 | 39 |
| Example 11 | R-11 | 50 | 3.2 | 30 |
| Example 12 | R-12 | 48 | 3.5 | 42 |
| Example 13 | R-13 | 53 | 2.9 | 39 |
| Example 14 | R-14 | 46 | 3.1 | 36 |
| Example 15 | R-15 | 49 | 3.0 | 31 |
| Example 16 | R-16 | 47 | 2.5 | 22 |
| Example 17 | R-17 | 45 | 2.7 | 20 |
| Example 18 | R-18 | 50 | 3.4 | 33 |
| Example 19 | R-19 | 55 | 3.2 | 41 |
| Example 20 | R-20 | 48 | 3.6 | 38 |
| Example 21 | R-21 | 49 | 3.5 | 31 |
| Example 22 | R-22 | 50 | 3.1 | 37 |
| Example 23 | R-23 | 49 | 3.2 | 42 |
| Example 24 | R-24 | 50 | 2.9 | 34 |
| Example 25 | R-25 | 48 | 3.7 | 46 |
| Example 26 | R-26 | 45 | 3.5 | 41 |
| Example 27 | R-27 | 48 | 3.1 | 46 |
| Example 28 | R-28 | 50 | 3.0 | 49 |
| Example 29 | R-29 | 49 | 3.2 | 39 |
| Comparative | CR-1 | 64 | 4.6 | 183 |
| Example 1 | | | | |
| Comparative | CR-2 | 68 | 4.3 | 271 |
| Example 2 | | | | |
| Comparative | CR-3 | 62 | 4.8 | 149 |
| Example 3 | | | | |
| Comparative | CR-4 | 66 | 4.8 | 328 |
| Example 4 | | | | |

As is clear from Table 4, all of the radiation-sensitive resin compositions of Examples 1 to 29 exhibited a sensitivity, an LWR performance, and a failure density, which were excellent, as compared with the radiation-sensitive resin compositions of Comparative Examples 1 to 4.

### <Formation of Resist Pattern (2)>

A 12-inch silicon wafer having an under-layer film having a thickness of 105 nm (ARC66, product of Brewer Science, Inc.) was used. Onto the surface of the wafer, each of the above-prepared radiation-sensitive resin compositions (R-30), (R-31), and (CR-5) was applied. The wafer was subjected to soft baking (SB) at 90° C. for 60 seconds and then cooled at 23° C. for 30 seconds, to thereby form a resist film having a thickness of 90 nm. Subsequently, the resist film was irradiated with light by means of an ArF excimer laser immersion lithography apparatus (NSR-S610C, product of NIKON, optical conditions: NA=1.3 and dipole (sigma 0.977/0.782)). After the light exposure, PEB was performed at 90° C. for 60 seconds and then, alkali development was performed by use of 2.38 mass % aqueous TMAH. The developed product was washed with water and dried, to thereby form a positive-type line-and-space pattern (pitch: 80 nm, line: 40 nm).

### <Evaluation of ArF Resist>

Physical properties of each of the resist patterns formed in the aforementioned <Formation of resist pattern (2)> was measured through the following procedures, to thereby evaluate the sensitivity of each radiation-sensitive resin composition, LWR performance, and failure density. Table 5 shows the results.

### [Sensitivity]

In the aforementioned <Formation of resist pattern (2)>, a dose which can form a line pattern having a width of 40 nm was employed as an optimum dose, serving as a sensitivity index (mJ/cm²). A sensitivity index lower than 25 mJ/cm² was evaluated as “good”, and a sensitivity index of 25 mJ/cm² or higher was evaluated as “bad”.

### [LWR Performance]

Through the same procedure as employed in the aforementioned <Evaluation of EUV resist>, the variation in resist pattern width (3(7) was determined. LWR performance was evaluated on the basis of the same ratings.

### [Failure Density]

The above-formed positive-type line-and-space pattern (pitch: 80 nm, line: 40 nm) was subjected to a failure inspection by means of KLA2925 (product of KLA). The smaller the measurement, the more excellent the failure density. The failure density was evaluated as “good” when the measurement was less than 50 ea/cm², and as “bad” when the measurement was 50 ea/cm² or more.

TABLE 5

| | Radiation- | | | Failure |
|---|---|---|---|---|
| | sensitive resin | Sensitivity | LWR | density |
| | composition | (mJ/cm²) | (nm) | (ea/cm²) |
| Example 30 | R-30 | 23 | 3.4 | 18 |
| Example 31 | R-31 | 20 | 3.6 | 21 |
| Comparative | CR-5 | 28 | 4.3 | 125 |
| Example 5 | | | | |

As is clear from Table 5, all of the radiation-sensitive resin compositions of Examples 30 and 31 exhibited a sensitivity, an LWR performance, and a failure density, which were excellent, as compared with the radiation-sensitive resin composition of Comparative Example 5.

The test results have revealed that the radiation-sensitive composition and the resist pattern formation method of the present disclosure can improve sensitivity, LWR performance, and failure density, as compared with those hitherto attained. Therefore, the radiation-sensitive composition and the resist pattern formation method of the present disclosure are suited for forming fine resist patterns in a lithography step for fabricating various electronic devices including semiconductor devices and liquid crystal devices.

1: A radiation-sensitive composition comprising:
a polymer comprising an acid-releasable group; and
at least one compound (b) selected from the group consisting of a compound represented by formula (1) and a compound represented by formula (2); 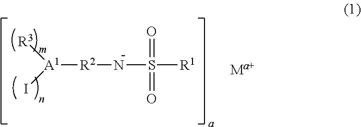
wherein, in the formula (1), R¹ represents a C1 to C20 monovalent organic group; R² represents a single bond or a C1 to C20 divalent group which bonds to N⁻ in the formula (1) via —CR⁴R⁵— or an aromatic ring; each of R⁴ and R⁵ independently represents a hydrogen atom, a C1 to C3 monovalent hydrocarbon group, or —COOR⁶; R⁶ represents a C1 to C6 monovalent hydrocarbon group; A¹ represents a group formed by removing (m+n+1) hydrogen atoms from an aromatic ring; R³ represents a monovalent substituent of A¹ other than an iodine atom; m is an integer of 0 or greater; n is an integer of 1 or greater; when m is ≥2, a plurality of R³s are identical to or different from one another; M^{a+} represents an a-valent cation; and a is 1 or 2; 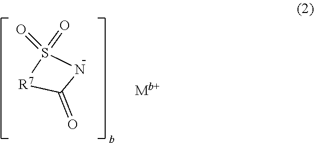
wherein, in the formula (2), R⁷ represents a group represented by formula (r-1), a group represented by formula (r-2), or a group represented by formula (r-3); M^{b+} represents a b-valent cation; and b is 1 or 2; 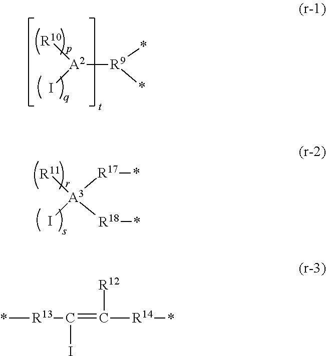
wherein, in formula (r-1), R⁹ represents a C1 to C10 (t+2)-valent hydrocarbon group, a C1 to C10 (t+2)-valent organic group formed by replacing any methylene group of the hydrocarbon group with —O—, —S—, —NR¹⁵—, or a carbonyl group, or a (t+2)-valent group formed by substituting any hydrogen atom of the hydrocarbon group or the (t+2)-valent organic group with a substituent; R¹⁵ represents a hydrogen atom or a C1 to C5 monovalent hydrocarbon group; A² represents a group formed by removing (p+q+1) hydrogen atoms from an aromatic ring; R¹⁰ represents a monovalent substituent of A² other than an iodine atom; p is an integer of 0 or greater; q is an integer of 1 or greater; when p is ≥2, a plurality of R¹⁰s are identical to or different from one another; t is 1 or 2; when t is 2, a plurality of A²s are identical to or different from one another, and a plurality of groups R¹⁰s are identical to or different from one another; and * represents a chemical bond;
in formula (r-2), A³ represents a group formed by removing (r+s+2) hydrogen atoms from an aromatic ring; R¹¹ represents a monovalent substituent of A³ other than an iodine atom; each of R¹⁷ and R¹⁸ independently represents a single bond, —O—, —S—, —NR¹⁹—, a carbonyl group, a C1 to C3 divalent chain hydrocarbon group, or a C1 to C6 divalent group formed by replacing any methylene group of the chain hydrocarbon group with —O—, —S—, —NR¹⁹—, or a carbonyl group; R¹⁹ represents a hydrogen atom or a C1 to C5 monovalent hydrocarbon group; r is an integer of 0 or greater; s is an integer of 1 or greater; when r is ≥2, a plurality of groups R¹¹s are identical to or different from one another; and * represents a chemical bond; and
in formula (r-3), R¹² represents a hydrogen atom, an iodine atom, or a C1 to C5 monovalent hydrocarbon group; each of R¹³ and R¹⁴ independently represents a single bond, —O—, —S—, —NR¹⁶—, a carbonyl group, a C1 to C3 divalent chain hydrocarbon group, or a C1 to C6 divalent group formed by replacing any methylene group of the chain hydrocarbon group with —O—, —S—, —NR¹⁶—, or a carbonyl group; R¹⁶ represents a hydrogen atom or a C1 to C5 monovalent hydrocarbon group; and *represents a chemical bond.

2: The radiation-sensitive composition according to claim 1, wherein R² is a single bond or a divalent group represented by formula (3);
—R²⁰—R²¹—*¹ (3)
wherein, in the formula (3), R²⁰ represents a single bond or a divalent linking group; R²¹ represents —CR⁴R⁵— or a divalent aromatic ring group; R⁴ and R⁵ have the same definitions as those of the aforementioned formula (1); and *¹ represents a chemical bond bound to N⁻ in the formula (1).

3: The radiation-sensitive composition according to claim 1, wherein the polymer comprises a structural unit which comprises an aromatic ring and a hydroxy group bonded to the aromatic ring.

4: The radiation-sensitive composition according to claim 1, wherein the compound (b) is a compound which can generates an acid in the radiation-sensitive composition upon irradiation with a radiation, and
the composition further comprises a compound which can generate an acid stronger than the compound (b) in the radiation-sensitive composition upon irradiation with the radiation.

5: A resist pattern formation method, comprising:
forming a resist film on a substrate by applying the radiation-sensitive composition according to claim 1;
exposing the resist film to a radiation; and
developing the radiation-exposed resist film.

6-7. (canceled)

8: A compound represented by formula (1): 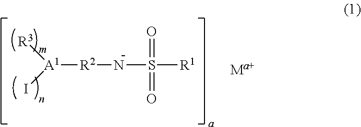
wherein, in the formula (1), R¹ represents a C1 to C20 monovalent organic group; R² represents a single bond or a C1 to C20 divalent group which bonds to N⁻ in the formula (1) via —CR⁴R⁵— or an aromatic ring; each of R⁴ and R⁵ independently represents a hydrogen atom, a C1 to C3 monovalent hydrocarbon group, or —COOR⁶; R⁶ represents a C1 to C6 monovalent hydrocarbon group; A¹ represents a group formed by removing (m+n+1) hydrogen atoms from an aromatic ring; R³ represents a monovalent substituent of A¹ other than an iodine atom; m is an integer of 0 or greater; n is an integer of 1 or greater; when m is ≥2, a plurality of R³s are identical to or different from one another; M^{a+} represents an a-valent cation; and a is 1 or 2.

9: A compound represented by formula (2); 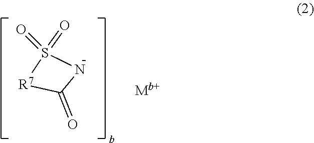
wherein, in the formula (2), R⁷ represents a group represented by the formula (r-1), a group represented by formula (r-2), or a group represented by formula (r-3); M^{b+} represents a b-valent cation; and b is 1 or 2; 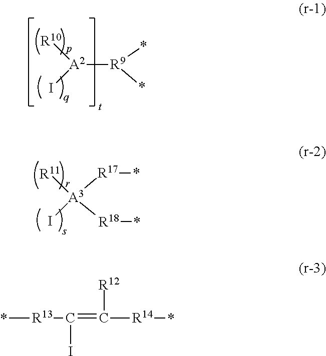
wherein, in formula (r-1), R⁹ represents a C1 to C10 (t+2)-valent hydrocarbon group, a C1 to C10 (t+2)-valent organic group formed by replacing any methylene group of the hydrocarbon group with —O—, —S—, —NR¹⁵—, or a carbonyl group, or a (t+2)-valent group formed by substituting any hydrogen atom of the hydrocarbon group or the (t+2)-valent organic group with a substituent; R¹⁵ represents a hydrogen atom or a C1 to C5 monovalent hydrocarbon group; A² represents a group formed by removing (p+q+1) hydrogen atoms from an aromatic ring; R¹⁰ represents a monovalent substituent of A² other than an iodine atom; p is an integer of 0 or greater; q is an integer of 1 or greater; when p is ≥2, a plurality of R¹⁰s are identical to or different from one another; t is 1 or 2; when t is 2, a plurality of A²s are identical to or different from one another, and a plurality of groups R¹⁰s are identical to or different from one another; and * represents a chemical bond;
in formula (r-2), A³ represents a group formed by removing (r+s+2) hydrogen atoms from an aromatic ring; R¹¹ represents a monovalent substituent of A³ other than an iodine atom; each of R¹⁷ and R¹⁸ independently represents a single bond, —O—, —S—, —NR¹⁹—, a carbonyl group, a C1 to C3 divalent chain hydrocarbon group, or a C1 to C6 divalent group formed by replacing any methylene group of the chain hydrocarbon group with —O—, —S—, —NR¹⁹—, or a carbonyl group; R¹⁹ represents a hydrogen atom or a C1 to C5 monovalent hydrocarbon group; r is an integer of 0 or greater; s is an integer of 1 or greater; when r is ≥2, a plurality of groups R¹¹s are identical to or different from one another; and * represents a chemical bond; and
in formula (r-3), R¹² represents a hydrogen atom, an iodine atom, or a C1 to C5 monovalent hydrocarbon group; each of R¹³ and R¹⁴ independently represents a single bond, —O—, —S—, —NR¹⁶—, a carbonyl group, a C1 to C3 divalent chain hydrocarbon group, or a C1 to C6 divalent group formed by replacing any methylene group of the chain hydrocarbon group with —O—, —S—, —NR¹⁶—, or a carbonyl group; R¹⁶ represents a hydrogen atom or a C1 to C5 monovalent hydrocarbon group; and *represents a chemical bond.

10: A method of generating an acid, comprising irradiating the compound of claim 8 with a radiation.

11: A method of generating an acid, comprising irradiating the compound of claim 9 with a radiation.

12: The radiation-sensitive composition according to claim 1, wherein M^{a+} represents at least one cation selected from the group consisting of a sulfonium cation and, an iodonium cation.

13: The radiation-sensitive composition according to claim 1, wherein M^{a+} and M^{b+} each independently represent at least one cation selected from the group consisting of a sulfonium cation and, an iodonium cation.

14: The radiation-sensitive composition according to claim 1, wherein in the formula (2), R⁷ represents a group represented by formula (r-1) or a group represented by formula (r-2).

15: The radiation-sensitive composition according to claim 12, wherein in the formula (2), R⁷ represents a group represented by formula (r-1) or a group represented by formula (r-2).
